(19) United States
(12) Patent Application Publication
(10) Pub. No.: US 2025/0387577 A1
(43) Pub. Date: Dec. 25, 2025

(54) Multiple Gas Supply Insufflator System and Method

(71) Applicant: Lexion Medical, LLC, St. Paul, MN (US)

(72) Inventors: Carl M. Geisz, Edina, MN (US); Samuel Porter, Apple Valley, MN (US)

(21) Appl. No.: 19/244,409

(22) Filed: Jun. 20, 2025

Related U.S. Application Data
(60) Provisional application No. 63/663,776, filed on Jun. 25, 2024.

(51) Int. Cl. A61M 13/00

(52) U.S. Cl. CPC A61M 13/003; A61M 2205/3344

(57) ABSTRACT

One aspect of the invention is an insufflator having at least a first and second gas input connection, The first and second gas input connections are operable to be connected to a first gas source and a second gas source, respectively. The insufflator has a first gas output connection. A first flow regulation unit may be coupled to the first and second gas input connection and to the first gas output. The first flow regulation unit may be configured to regulate the flow of gas through the first gas output connection in response to pressure measurements equal or proportional to the pressure in a body cavity to which the insufflator is supplying gas.

### RELATED APPLICATIONS

This application claims the priority benefit of U.S. Provisional Application No. 63/663,776, filed Jun. 25, 2024 and entitled, “Multiple Gas Supply Insufflator System and Method,” which is incorporated by reference in: its entirety.

### TECHNICAL FIELD

The present disclosure relates generally to minimally invasive surgery and more particularly to an insufflation system and method.

### BACKGROUND

It is common in forms of minimally invasive surgery to inflate a body cavity. For example, in various minimally invasive procedures, the peritoneal cavity is inflated to provide working space for the surgeon. The peritoneal cavity is typically inflated using an insufflator that delivers carbon dioxide gas to the peritoneal cavity and seeks to maintain a stable pressure while the surgery is being performed.

In most cases, surgeons prefer for pressure in the body cavity to remain as stable as possible. Stable pressure may be more difficult to maintain for an insufflator where there are large leaks of insufflation gas out of the body cavity. Certain surgical procedure are prone to large leaks. Other surgical procedures, for example procedures that use a large number of trocars, are prone to a collection of small leaks that collectively challenge an insufflator to maintain a stable pressure.

Many commonly used insufflators are not capable of supplying the amount of gas necessary to maintain stable pressure inside the body cavity when a large amount of gas is leaking out of the abdomen. Many commonly used insufflators supply gas at 20 lpm or less to a body cavity. This can be problematic in procedures involving large leaks (or a collection of small leaks) that demand flows of 30 lpm or more to maintain stable pressure.

There is also a trend in minimally invasive surgery to use smaller diameter trocars so that a patient has smaller incisions. As trocar diameters get smaller, there is another challenge to maintaining stable pressure inside of the abdomen. Smaller diameter trocars restrict gas flow and often cannot supply even 20 lpm to the abdomen even if the insufflator is capable of supplying gas at such flow rates. Thus, both insufflator flow capacity and trocar flow restrictions make it challenging to maintain a stable pressure inside of a body cavity.

Accordingly, there is a need for an insufflation method and system that can reliably supply high volumes of gas to a body cavity.

### SUMMARY

According to one embodiment, an insufflator includes at least a first gas supply and a second gas supply. The first gas supply is coupled to a first flow regulation unit. The second gas supply is coupled to a second flow regulation unit. The first flow regulation unit is coupled to a first gas outlet while the second flow regulation unit is coupled to a second gas outlet.

The teachings of the disclosure provide one or more technical advantages. Embodiments of the disclosure may have none, some, or all of these advantages. For example, in one embodiment, the first gas outlet can be connected via tubing to a first trocar and the second gas outlet can be connected via separate tubing (which may or may not be joined to the other tubing) to a second trocar. This arrangement provides the potential to deliver twice the amount of gas to a body cavity than with use of a single channel, depending upon the flow restrictions in the tubing and trocar (or other delivery device) used.

Bischof has proposed in U.S. Published Patent Application 2021/0213214 to potentially deliver twice the amount of gas by using two pressure and flow regulation units connected to two trocars. However as shown in Bischof FIG. 1, a single gas supply line is used, unlike the present disclosure which may use two or more gas supply lines. This is significant because Bishof indicates the desire to provide flows on the order of 40-60 LPM. (Bischof, § 12). Flows in that range will lead to freeze-up problems when carbon dioxide (or other gas) is supplied with bottled gas. Gas cylinders, particularly those with a smaller mass, may cool so rapidly due to decompression of large volumes of gas at high flow rates that some of the gas may liquify or even solidify. Applicant is unaware of any published studies on the threshold where freeze-up may occur and of course this can depend on many variables such as room temperature, the thermal mass of the tank, etc. However, Applicant believes that freeze-up may occur with flows as low as 25 LPM. With increasing demand for high flow insufflators, the potential for freeze-up when using tanks to deliver insufflation gas is a problem to address.

Bottled CO2 typically is stored in pressurized metal tanks. As the gas leaves the tank to be supplied to the insufflator, it decompresses, which cools the gas as well as the tank. This is not an issue at flow rates around 20 lpm or lower. However, with flow rates in the 40-60 lpm range called for by Bischof, the cooling effect of decompression can lead to both liquification and solidification of the CO2. This can cause a surgery to need to be stopped while switching to a new bottle/tank. Often, a medical facility may discard the tank that froze up without using all of the gas in the tank because of a perception that something is wrong with the tank.

The invention greatly reduces the chance of freeze-up occurring when using bottles/tanks of a compressed gas such as CO2. By dividing the desired flow between two or more separate sources of gas, the gas provided by each gas source can be cut roughly by half or more, reducing the chance of freeze-up.

In some embodiments, the output the first flow regulation unit can be connected to the output of the second flow regulation unit such that the entire output of gas is supplied through the first gas outlet. By joining the output flows together, a high volume flow can be provided through a single trocar, provided that the trocar does not restrict the flow to prevent a desired throughput of gas (typically measured in liters per minute). If a trocar is being used that allows sufficient throughput of gas then this may avoid the need for the use of a second trocar, thus reducing the number of incisions in the patient.

The ability in some embodiments to connect to the outputs of both the first and second flow regulation units may have other advantages. For example, some have proposed using a mixture of two or more gasses for insufflation. Where a mixture of two or more gasses is desired, a first type of gas could be connected to the first gas supply line while a second type of gas could be connected to a second gas supply line. The user could then specify an approximate percentage of each gas to make up the total flow. The flow regulation units could then regulate the flow of each gas such that the combined output has the desired percentage mixture.

In some embodiments, the insufflator may have more than two gas supply connections, more than two flow regulation units, and more than two gas outlets. Each of the gas outlets may be connected to a trocar. The ability to achieve higher gas flows, whether using one or more trocars connected to one, two, or more outputs of the insufflator allows the insufflator to better adjust to higher flows demanded when large leaks out of a body cavity occur during certain surgeries. For example, a large leak occurs during a hysterectomy when the uterus is removed. The invention increases the chance that the surgery can continue without needing to stop (fully or partially) one or more leaks.

In robotic surgery in particular, the trend over the past several years has been to use trocars with smaller diameters. Smaller diameter trocars can be used with smaller incisions that allow a patient to heal more quickly. A problem with smaller diameter trocars for insufflation, however, is that the trocars restrict gas flow such that fewer liters per minute can pass through the trocar than are desired to maintain stable pneuoperitoneum, for example. The problem is exacerbated when the gas is flowing through the same lumen through which instruments are inserted through the trocar. Insertion of instruments further restricts flow and reduces the maximum number of liters per minute of gas that can flow through the trocar when gas delivery is through an instrument lumen.

For procedures where many small diameter trocars are used, embodiments of the disclosure may allow a total cumulative gas flow that is sufficient for most procedures, even those with large leaks. To provide that cumulative gas flow, one can simply use an embodiment of the disclosure that has two, three, four, or more gas supply lines, two three, four or more flow regulation units, and two, three, four or more gas outlets. Each of the gas outlets can be connected to a separate trocar. In some embodiments, a single gas outlet with high flow can be used but a tubing set that branches into multiple tubes and supplies multiple trocars can be used.

In some embodiments, there may be more flow regulation units and gas outlets than there are gas supply lines. For example, suppose that the flow is often limited by small diameter trocars to 10 liters a minute but it is desirable to have 40 liters a minute throughput. One would need four different trocars with each flowing 10 liters per minute to obtain the desired throughput. In this example, one might use two gas supply lines (each supplying 20 liters per minute approximately) and four flow regulation units and gas outlets for the four trocars. In such an embodiment, each of the gas supply lines would be coupled to two of the four flow regulation units. This embodiment would advantageously allow the desired flow to be achieved while reducing or eliminating the chances for freeze-up.

Embodiments of the disclosure may allow pressure sensing without fully interrupting the gas flow as is common with most conventional insufflators. In conventional insufflators, it is typical to cease gas flow (as flowing gas impacts pressure) so that the pressure at a sensor in the insufflator is approximately the same as the pressure in the abdomen. The problem with doing this, however, is that ceasing the flow of gas causes the pressure to drop due to any leaks that are occurring in the abdomen and the result is a somewhat oscillatory behavior of the pressure. Most surgeons would prefer for the pressure to remain as constant as possible. In some embodiments, pressure sensing may occur at one or more of the trocars (or other devices inserted into the body cavity) with a pressure sensor in or on the trocar (or other device). In such embodiments, pressure the sensor in the flow regulation unit in the insufflator may be used as a backup pressure sensor. Such an arrangement may eliminate the need to stop insufflation to measure pressure or greatly reduce the frequency of such stopping. In other embodiments, the flow of gas may be stopped for milliseconds out of one of the gas outlets to measure pressure while gas is still flowing through one or more other gas outlets. This would reduce the oscillatory problem as some gas would still be flowing through one or more trocars even while flow is stopped through one trocar.

### BRIEF DESCRIPTION OF THE DRAWINGS

For a more complete understanding of embodiments of the disclosure and the potential advantages thereof, reference is now made to the following written description taken in conjunction with the accompanying drawings, in which:

FIG. 1 illustrates an example embodiment of an insufflator with a single gas supply;

FIG. 2 illustrates an example embodiment of an insufflator with multiple gas supplies;

FIG. 3 illustrates another example embodiment of an insufflator with multiple gas supplies;

FIG. 4 illustrates another example embodiment of an insufflator with multiple gas supplies;

FIG. 5 illustrates another example embodiment of an insufflator with multiple gas supplies;

FIG. 6 illustrates another example embodiment of an insufflator with multiple gas supplies;

FIG. 7 illustrates another example embodiment of an insufflator with multiple gas supplies;

FIG. 8 illustrates another example embodiment of an insufflator with multiple gas supplies;

FIG. 9 illustrates another example embodiment of an insufflator with multiple gas supplies;

FIG. 10 illustrates another example embodiment of an insufflator with multiple gas supplies;

FIG. 11 illustrates another example embodiment of an insufflator with multiple gas supplies; and

FIG. 12 illustrates another example embodiment of an insufflator with multiple gas supplies.

### DETAILED DESCRIPTION OF THE DRAWINGS

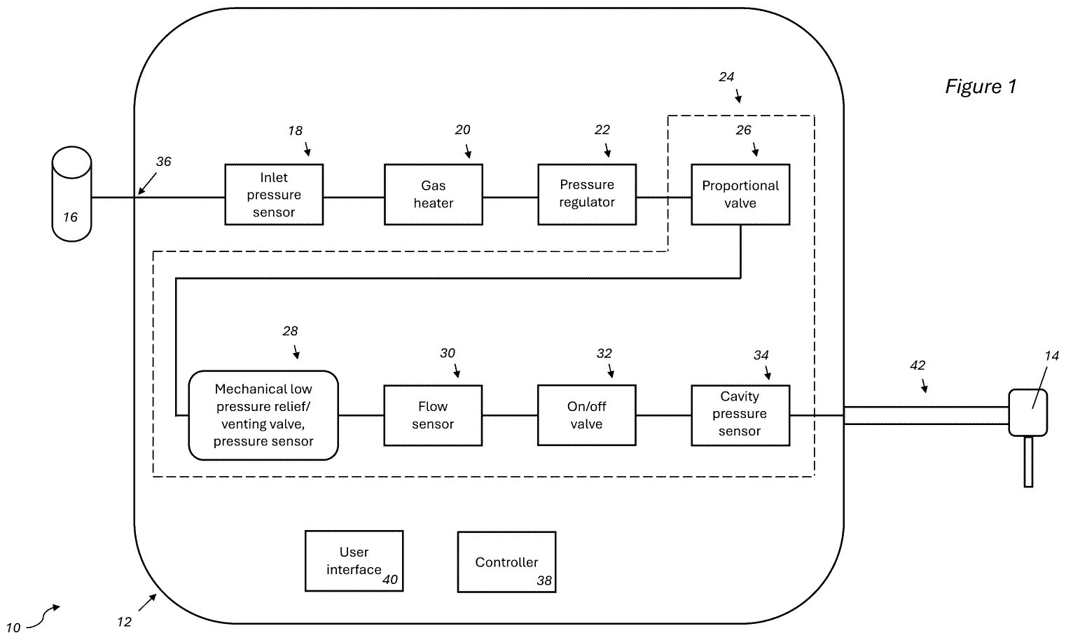

The teachings of certain portions of the present disclosure recognize certain benefits of an insufflator having multiple gas inputs. Example embodiments are best understood by referring to FIGS. 1 through 11 of the drawings and the description below, like numerals being used for like and corresponding parts of the various drawings.

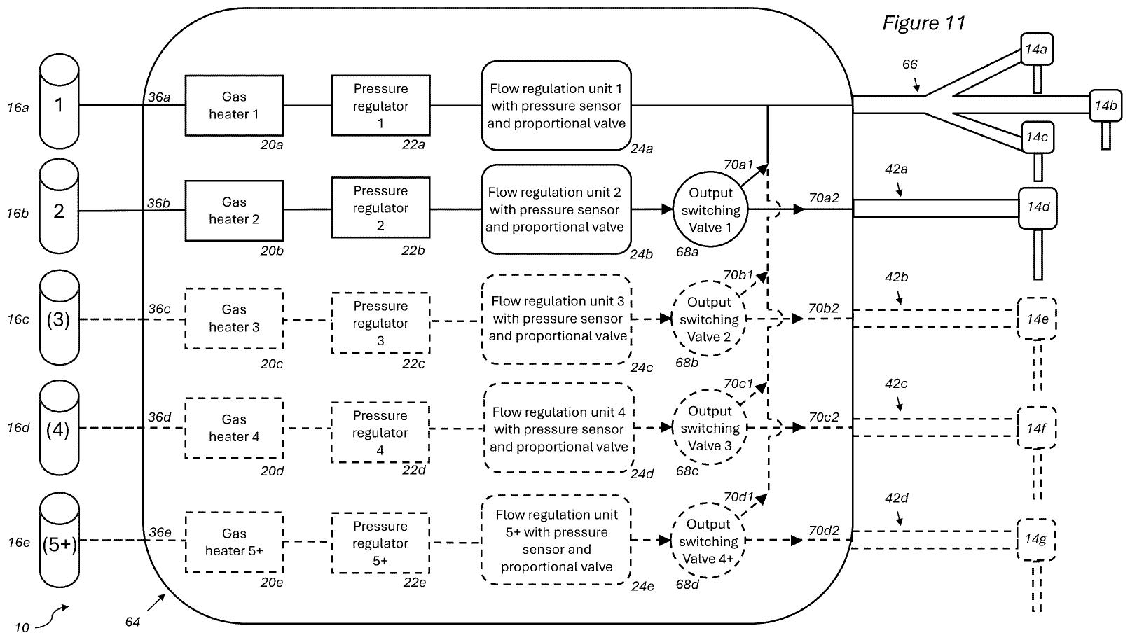

FIG. 1 illustrates an example embodiment of an insufflation system 10 comprising insufflator 12, trocar 14, and gas cylinder 16. Gas cylinder 16 may contain pressurized carbon dioxide or any other gas suitable for insufflation of a body cavity, such as helium. Trocar 14 is inserted into a body cavity and may be used to deliver insufflation gas from insufflator 12 to a body cavity. For example, trocar 14 may be used to deliver insufflation gas to the peritoneal cavity. One or more needles, trocars, robotic hubs, gel ports, or a colonoscope may be used in place of trocar 14 in FIG. 1 and in place of any trocar described in this patent. Insufflator 12 controls the delivery of gas to the body cavity with the goal of maintaining a stable pressure in the body cavity. Typically, it does so by using feedback control that seeks to keep the pressure inside the body cavity at a setpoint set by the user using user interface 40. A pressure sensor is used to sense a pressure equal or proportional to the pressure inside of the abdomen to enable feedback control. Insufflator 12 is typically connected to trocar 14 through tubing 42. Tubing 42 may include a filter (not explicitly shown) as is a common practice. Tubing 42 may also be a multipath tubing set like tubing set 66 illustrated in FIG. 11. A multipath tubing set may have 2, 3, or more branches off of a main conduit to supply insufflation gas to 2, 3, 4 of more gas delivery devices.

While insufflator 12 in this embodiment receives insufflation gas (here carbon dioxide) from a gas cylinder 16, it may also receive gas from a house supply in a medical facility that typically has a port on a wall that may be connected to insufflator 12 through gas connection 36. In this embodiment, gas cylinder 16 connects to insufflator 12 via gas connection 36. Gas connection 36 is typically a threaded metal connector. Various adapters (not explicitly shown) may be provided that connect into the threaded metal connector of gas connection 36 and that mate with whatever connector is attached to the hose running from gas cylinder 16 and/or the house gas supply in the medical facility. In some embodiments, the hose running from the cylinder 16 or house gas supply may be directly connected to the threaded metal connector. While a threaded metal connector is used in this embodiment, any suitable connector can be used to connect gas cylinder 16 (or the house supply or other supply) to insufflator 12.

While a single gas cylinder 16 is illustrated in FIG. 1, those skilled in the art will understand that multiple gas cylinders 16 may be used without departing from the scope of the invention. It is not unusual for a medical facility to connect multiple tanks to a splitting valve on a hose line such that medical personnel may select between one tank or the other using the splitting valve. When one tank gets low on gas, the splitting valve can be switched to the other tank so a medical procedure can continue while the tank is low or empty gets swapped out for a new tank with gas in it.

Optionally, an inlet pressure sensor 18 may be provided to sense the pressure of the gas as it enters insufflator 12. Inlet pressure sensor 18 may be electronically or wirelessly connected to controller 38 so that controller 38 receives an indication of the pressure that is sensed by pressure sensor 18. Inlet pressure sensor can be used for multiple purposes. First, inlet pressure sensor can be used to determine whether a house supply of insufflation gas (e.g. through a wall connection in a medical facility) or a gas cylinder is connected to insufflator 12 at gas connection 36. Typically, a gas cylinder will supply gas at 800-900 psi while a house connection will be somewhere between 30-100 psi. Various functions of the insufflator might vary depending upon which type of gas is supplied. For example, controller 38 may control gas heater 20 to supply less heat to heat the gas entering the insufflator when the gas supply is a house supply versus when the gas supply comes from a gas cylinder. Because of the high pressure the gas is under in the gas cylinder, decompression of that gas has a substantial cooling effect on the gas (and the cylinder) and more heat is typically required to heat the gas coming from a gas cylinder.

Second pressure sensor 18 can be used to make sure the gas supply is functioning properly. If the sensed pressure is too low or too high, controller 38 can generate a warning message to display on user interface 40 and can control insufflator 12 accordingly to be sure safe operation is maintained.

Third, pressure sensor 18 can be used to sense when a gas cylinder 16 is near empty. When controller 38 determines that the pressure has dropped below a specific threshold for a specific period of time based upon the output of pressure sensor 18, then controller 38 may generate a message (visual, audible, or both) or alarm to be output through user interface 40 to alert medical personnel that a gas cylinder 16 needs to be changed out.

Pressure sensor 18 can be an analog sensor or digital sensor. In the case of a digital sensor, the output data comprising the pressure readings may be supplied to controller 38 digitally. In the case of an analog sensor, an analog to digital converter (not explicitly shown) may be used to convert the analog signal indicating the pressure reading into a digital signal that can be used by controller 38.

Gas heater 20 may be used to heat the gas as it enters the insufflator or just inside the insufflator. In some embodiments, gas heater 20 may apply heat to gas connection 36 and heat up the metal fitting to heat the gas as it enters the insufflator. Gas heater 20 may be a resistive heater and may or may not include a temperature sensor. Gas heater may be controlled by controller 38. In some embodiments, controller 38 may simply turn gas heater 20 on or off. In other embodiments, controller 38 may adjust the amount of heat produced by gas heater 20. Gas heater 20 may be in various locations. In some embodiments, gas heater 20 may apply heat to pressure regulator 22. In other embodiments, gas heater 20 may be anywhere along the flow path between gas connection 36 and flow regulation unit 24. Gas heater 20 may heat up flow channels along this flow path in one or more locations. In some embodiments, gas heater 20 may be a unit that gas flows through on its way to flow regulation unit 24.

Pressure regulator 22 may be used to regulate the pressure of the gas such that it is at a lower pressure (e.g. about 35 psi) before entering flow regulation unit 24. Such a pressure regulator is commonly used in commercially available insufflators. A pressure regulator that regulates pressure to a different pressure may be used without departing from the scope of the invention. In some embodiments, pressure regulator 22 may be omitted or may be external to insufflator 12.

Flow regulation unit 24 is responsible for regulating the flow of gas to the body cavity. While one particular flow regulation unit 24 is illustrated in FIG. 1, different flow regulation units capable of controlling the flow of gas exiting insufflator 12 to supply to a body cavity may be used without departing from the scope of the invention. Based upon the desired pressure and flow settings that may be provided by a user through user interface 40, controller 38 may control flow regulation unit 24 such that the pressure inside of the body cavity is controlled within a reasonable range based upon a setpoint and such that gas flows at a particular rate. In some embodiments, the pressure in the body cavity may be regulated without any setting for flow rate.

Controller 38 may be a microprocessor or microcontroller that is running computer software stored in a memory. Controller 38 may receive electronic signals from one or more sensors of insufflator 12 and may control one or more valves of insufflator 12 based upon the readings of the sensors and one or more settings input by a user of the insufflator. User interface 40 may be used to receive input from a user of interface 40 and provide output to the user. User interface 40 may comprise a computer display or a touch screen or a series of visible electronic display components (e.g. alphanumeric LED modules). User interface 40 may further comprise electronic components to receive input from a user of insufflator 12 such as buttons, a touch screen, a bluetooth interface connecting to external controls (e.g. an I-phone app), a key board, a key pad, or a combination of any of the foregoing.

As will be understood by those in the art, flow regulation unit 24 may comprise a proportional valve 26, a pressure sensor and valving unit 28, a flow sensor 30, and on/off valve 32 and a cavity pressure sensor 34. Some or all of these components may be omitted without departing from the scope of the invention. Proportional valve 26 controls the flow of gas very precisely and may be controlled by controller 38 and appropriate software as is understood in the art. Proportional valve 26 is controlled in this embodiment based upon outputs of flow sensor 30 and a pressure sensor that is in pressure sensor and valving unit 28. Feedback of pressure and flow on the downstream side of the proportional valve may be used to control the proportional valve to obtain the desired pressure and flow conditions for gas exiting insufflator 12. The location of the pressure and flow sensors may vary without departing from the scope of the invention. While one embodiment of flow regulation unit 24 is shown in FIG. 1, any type of flow regulation unit 24 capable of controlling flow to seek to maintain a body cavity at a pressure setpoint may be used without departing from the scope of the invention. Thus, the flow regulation unit 24 in the rest of the Figures of this patent may have the same or different structure than the flow regulation unit illustrated in FIG. 1.

In this embodiment, a pressure sensor is part of a pressure sensor and valving unit 28 but could be a separate component. Pressure sensor and valving unit 28 also comprises a mechanical low pressure relief valve and a venting valve. These components could also be separate components or other components could be part of the pressure sensor and valving unit 28. Pressure sensor and valving unit 28 comprises a mechanical low pressure relief valve that may be used to protect the patient from high pressure buildup inside of a body cavity in the event of a malfunction. This valve may open when the pressure exceeds a threshold and vent gas to the atmosphere out of insufflator 12 in order to relieve pressure. In this embodiment, the threshold for venting to occur is 85 mm of mercury. Pressure sensor and valving unit 28 may also comprise a vent valve that may be used to vent pressure to the atmosphere if insufflator 12 determines that pressure is too high inside of the body cavity. For example if someone were to press down continuously on the abdomen during a laparoscopic procedure, the pressure inside of the abdomen could be kept at a safer level by venting gas using the vent valve.

On/off valve 32 may be used to turn the flow of gas out of insufflator 12 on or off. In some embodiments, this valve may be electrically controlled using controller 38.

Cavity pressure sensor 34 is optional and may or may not be included in insufflator 12. In this embodiment, cavity pressure sensor 34 can be used to sense the pressure inside of the body cavity when gas flow is temporarily stopped to allow for the pressure measurement. Flowing gas will interfere with the ability to obtain an accurate flow measurement. In some embodiments, insufflation is ceased for less than a second in order to conduct a pressure measurement. In other embodiments, a dedicated flow path (e.g. using a tube) may run from cavity pressure sensor 34 to a location that is at approximately the same pressure as the body cavity—e.g. within a chamber of a multi-lumen trocar that does not have gas flowing through the chamber.

The gas supplied by insufflator 12 may be transported to a body cavity via tubing 42 and trocar 14. Trocar 14 could be replaced with a needle, robotic hub, gel port, colonoscope, or any other type of gas delivery device used to deliver gas to a body cavity. Insufflator 12 may be used with any device that can deliver gas to a body cavity.

There are numerous options for trocar 14. Trocar 14 may be a multi-lumen trocar with one or more pressure sensors on or inside Trocar 14. In some cases, the multi-lumen trocar can have multiple chambers within an annular lumen. One of those chambers can be used for gas flow while the other is used for pressure sensing. Another lumen can be used for instruments used in a surgical procedure to pass through. In some embodiments, one or more pressure sensors may be on the outside of the trocar or enclosed within elastomeric seals that are a part of the trocar. Trocar 14 may have a heater and an absorbent material or reservoir so that insufflation gas may be heated and/or humidified. Trocar 14 may also have a temperature sensor and a humidity sensor. The temperature sensor may be used to control the heater temperature-either under control of circuitry within trocar 14 or under control of controller 38. The humidity sensor may be used to generate a message or alarm (either visible or audible or both) using user interface 40 so that medical personnel may be alerted to add more water to trocar 14.

In some embodiments, one or more pressure sensors will be in or on trocar 14 but insufflator 12 will still have pressure sensor 34. In such embodiments, pressure sensor 34 can be used in case of a malfunction of a sensor within trocar 14 and/or to test whether the sensor in the trocar (or other gas delivery device) 14 is working properly. In such embodiments, insufflation may not need to be stopped to make a measurement using pressure sensor 34 nearly as often as when there is no sensor within the trocar.

Example trocars or needles that can be used in insufflation system 10 and techniques for using them can be found in the following United States Patent Applications, each of which is incorporated by reference as if fully set forth herein: (1) U.S. patent application Ser. No. 16/592,358, which was published on Apr. 8, 2021, in publication number US 2021-0100964 and is entitled, Method and System for Delivering Insufflation fluid, (2) U.S. patent application Ser. No. 18/337,849, which was filed on Jun. 20, 2023 and is entitled, Method and system for insufflating a body cavity using a percutaneous needle, (3) U.S. patent application Ser. No. 13/065,438, filed on Mar. 22, 2011, and entitled Insufflation Apparatus, (4) U.S. patent application Ser. No. 15/610,026, filed on May 31, 2017 and entitled Method and system for controlling pressurization of a patient cavity using a pressure sensor in a trocar, (5) U.S. patent application Ser. No. 16/570,685, which was published on Jan. 2, 2020 in publication number US 2020-0001025, and entitled Method and system for measuring pressure in a body cavity, (6) U.S. patent application Ser. No. 16/206,284, which was published on Mar. 28, 2019 in publication number US 2019/0091421, and is entitled Method and system for controlling pressurization of a patient cavity using a pressure sensor of a medical appliance, (7) U.S. patent application Ser. No. 16/264,011, which was published on Jun. 13, 2019 in publication number US 2019/0175213, and is entitled Method and system for measuring pressure in a body cavity, (8) U.S. patent application Ser. No. 16/271,072, which was published on Jun. 6, 2019 in publication number US 2019/0167301, and is entitled Method and system for gas maintenance to a body cavity using a trocar, (9) U.S. patent application Ser. No. 15/293,013, which was patented as U.S. Pat. No. 10, 835, 284, and entitled Method and system for controlling pressurization of a patient cavity using cavity distension measured by a pressure sensor of a trocar, (10) U.S. patent application Ser. No. 15/251,511, which was patented as U.S. Pat. No. 10,595,897, and entitled Method and system for measuring pressure in a body cavity using a trocar, and (11) U.S. patent application Ser. No. 14/792,873, which was patented as U.S. Pat. No. 10,238,421 and entitled Method and system for gas maintenance to a body cavity using a trocar. In addition, trocars typically used in robotic systems that may provide a simple luer connection that is connected via a flow path to a lumen of the trocar may also be used. Any of these trocars, needles, robotic hubs, gel ports, colonoscopes, and techniques and methods can be used with any of the embodiments herein.

Note that the location of various components for insufflator 12 may be rearranged without departing from the scope of the invention. Also, various components may be deleted and/or other components added without departing from the scope of the invention.

In modern laparoscopic procedures, there are multiple ways that insufflation gas can leak outside of a body cavity. First, the surgical procedure may create a leak, such as in a hysterectomy when the uterus is removed. Second, gas can leak around the edges of a trocar through the incision in the patient. Third, gas can leak around seals in trocars or needles as instruments are inserted, removed, or manipulated. Fourth, gas can be evacuated when suction is used or when smoke is evacuated from a body cavity.

In some procedures, the amount of gas leaking or being sucked out of a body cavity is substantial. Many existing insufflators cannot keep up with the leaks. Most insufflators deliver 15-20 lpm of insufflation gas maximum. However, due to leaks or gas being sucked out of the abdomen, surgeons may desire gas flows between 30 and 60 lpm or higher in some cases. At these flow rates, however, if carbon dioxide (or other gas) cylinders are used as the source of insufflation gas, there is a substantial risk of a freeze-up condition occurring. Due to the cooling of the carbon dioxide tank from the decompression occurring at these flow rates, the carbon dioxide can liquify and even solidify. This can cause malfunctions of the insufflator and can greatly reduce the flow of insufflation gas at the very time when the need for high flows is greatest. Embodiments of the invention reduce or eliminate the likelihood of freeze-up occurring when gas cylinders are used with high gas flows.

The embodiments herein are shown as operating using gas cylinders as a source of gas. In most cases, carbon dioxide cylinders will be used. Other types of gases can be used without departing from the scope of the invention. In addition, a house connection, usually to a port on a wall of a medical facility operable to supply gas to the insufflator can be used. A conduit (e.g. a hose or tubing) connected to a house source of insufflation gas can be split into multiple conduits to connect to multiple inputs of the insufflators disclosed herein.

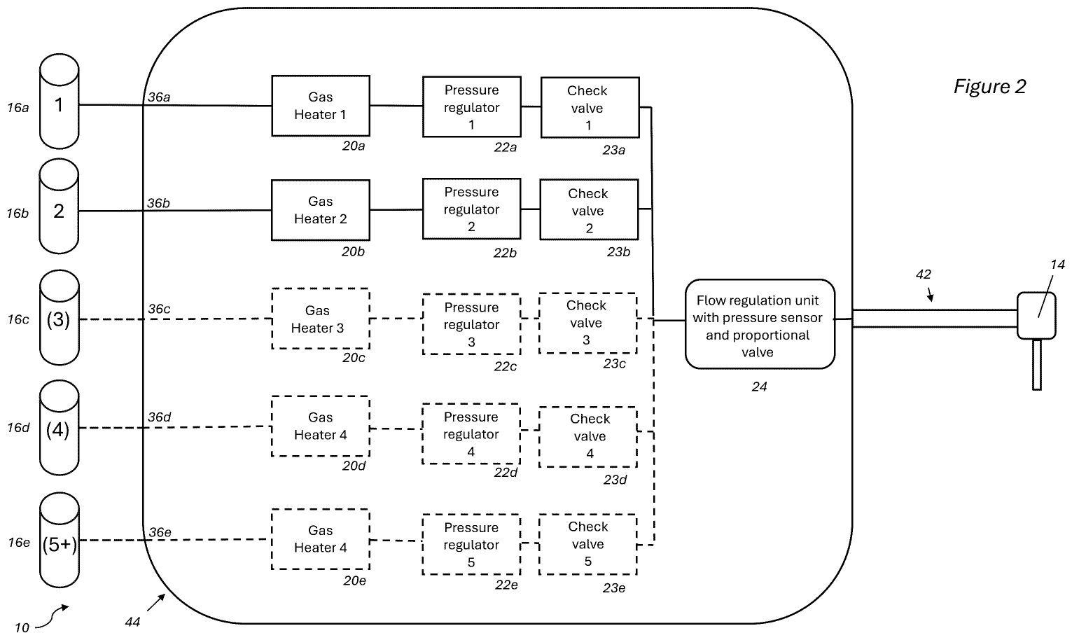

FIG. 2 illustrates an example insufflator 44 that can be used to provide high gas flows while reducing or eliminating the risk of freeze-up. In this embodiment, two gas cylinders, 16a and 16b are connected to the insufflator. Additional gas cylinders 16c-16e are illustrated to show that this architecture for insufflator 44 could be extended to attach to any number of cylinders. Also, as discussed above in connection with FIG. 1, each illustrated gas cylinder could instead be multiple gas cylinders connected to a splitting valve. Also, while not explicitly shown each flow path from a tank through a pressure regulator 22 may also include an inlet pressure sensor 18 that may be used as described above in connection with FIG. 1. Each flow path from a tank through a pressure regulator 22a-22e may also include a gas heater 20a-20e. In some embodiments, a single heater may be used to heat more than one gas conduit that is fluidly connected to one of the gas cylinders 16a-16e. Of course, a gas heater 20A-20E may be configured in any of the ways discussed in connection with FIG. 1 or otherwise. For example, the heater may heat up the gas connection 36A-36E, be a stand-alone piece of the flow path, or heat the gas within pressure regulators 22a-22e. All of the options discussed in connection with FIG. 1 can be used for the gas connections 36a-36e in FIG. 2.

In this embodiment, the gas flow from multiple cylinders is combined into a flow for output to a single gas delivery device such as trocar 14. All of the trocar options (or other gas delivery device options) discussed above in connection with FIG. 1 may be used in connection with insufflator 44 of FIG. 2 in place of the illustrated trocar 14. Flow regulation unit 24 is used to regulate the flow of insufflation gas through tubing 42 to trocar 14 to attempt to maintain pressure inside of a body cavity at a constant setpoint set by medical personnel during the use of insufflator 44.

As illustrated, the flow from multiple gas cylinders 16a-16e (which could be 2 or any other number of cylinders) is combined using conduits with T-junctions to join the flows together into a single flow into flow regulation unit 24. Other types of conduits could be used other than T-junctions to join the flows together without departing from the scope of the invention. (As discussed above, the gas sources could also be something other than cylinders, such as a house supply of gas.” This embodiment reduces the risk of freeze-up by dividing the high gas flow demands among multiple cylinders. Where two cylinders are used, approximately half of the gas flow will come from each cylinder, thus reducing cooling in each cylinder due to reduced gas flow demands.

An input pressure sensor 18a-18e (not explicitly shown) in each flow path may alert insufflator 44 when one or more gas cylinders 16a-16e is not connected to the insufflator. When insufflator 44 detects a missing gas cylinder (or other gas supply), it may close a valve 23a-23e to prevent gas that is flowing into insufflator 44 from one of the gas cylinders 16a-16e to be discharged out of one of the gas connections 36a-36e where no gas cylinder is connected. Valves 23a-23e may also be check valves that operate automatically to prevent flow back out of one of the gas connections 36a-36e. The valves 23a-23e could be located anywhere along the flow path between the gas connections 36a-36e and where the flows from multiple connections are joined together.

While not explicitly shown, this insufflator 44 and all insufflators shown in the remaining figures may have a controller 38 and user interface 40 as described above in connection with FIG. 1.

In operation, two or more gas cylinders, 16a-16e are connected to one of the gas connections 36a-36e (one for each cylinder) for insufflator 44. When the insufflator is in operation, each cylinder 16a-16e has its own flow channel through a regulator 22a-22e. The gas in each flow path is heated by at least one of the gas heaters 20A-20E. The outputs of the pressure regulators 22A-22E are combined into single flow that runs through a conduit into flow regulation unit 24. Flow regulation unit 24 regulates the flow characteristics of the gas and seeks to maintain via feedback control a relatively constant pressure within a body cavity into which trocar 14 is inserted (e.g. the peritoneal cavity). It seeks to maintain the pressure at a setpoint that is established by a user. Again, all of the trocar (or other gas delivery device) options set forth in connection with FIG. 1 may be used for trocar 14 and the techniques for using such trocars (or other devices) and operating insufflator 44 discussed in connection with FIG. 1 may also be used.

In operation, two or more gas cylinders, 16a-16e are connected to one of the gas connections 36a-36e (one for each cylinder) for insufflator 44. When the insufflator is in operation, each cylinder 16a-16e has its own flow channel through a pressure regulator 22a-22e up to a T-junction, after which the flows are combined to flow into flow regulation unit 24. The conduits on the output side of the pressure regulators can be joined in any suitable manner to join the flows from each of the operation gas cylinders 16a-16e (or other type of gas supply such as a house supply). The gas in each flow path may be heated by at least one of the gas heaters 20A-20E. The flow from the cylinders is combined into single flow that runs through a conduit into flow regulation unit 24. Flow regulation unit 24 regulates the flow characteristics of the gas so as to maintain a relatively constant pressure within a body cavity into which trocar 14 (or other gas delivery device) is inserted (e.g. the peritoneal cavity). As discussed above, flow regulation unit 24 regulates the flow of gas based upon feedback control using pressure sensors to sense a pressure equal or proportional to the pressure inside of the body cavity and attempts to flow gas (or relieve pressure) so as to keep the pressure close to a pressure setpoint set by medical personnel. Again, all of the trocar (or other gas delivery device) options set forth in connection with FIG. 1 may be used for trocar 14 and the techniques for using such trocars and operating insufflator 44 discussed in connection with FIG. 1 may also be used.

Persons skilled in the art will understand that each of the lines connecting various components in insufflator 44 comprise conduits to create a flow path for gas from one component to another. These conduits can be tubing, channels within a solid material, a combination of different types of conduits, etc.

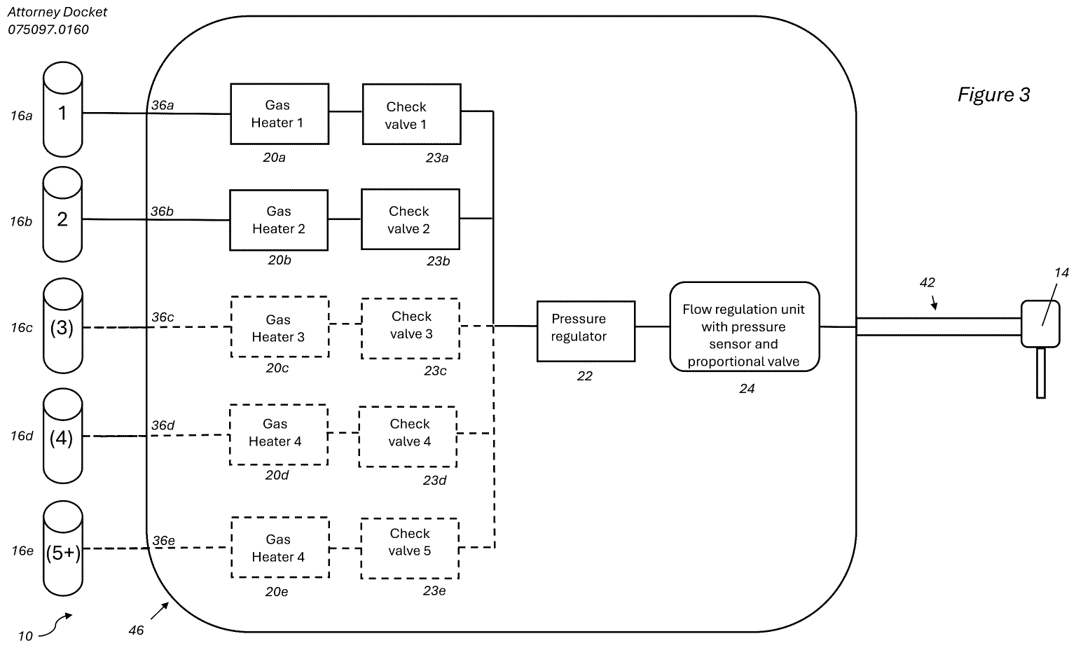

FIG. 3 illustrates another example insufflator 46 that can be used to provide high gas flows while reducing or eliminating the risk of freeze-up. In this embodiment, at least two gas cylinders, 16a and 16b are connected to the insufflator. Additional gas cylinders 16c-16e are illustrated to show that this architecture for insufflator 46 could be extended to attach to any number of cylinders. The five cylinders shown are only an example. Also, as discussed above in connection with FIG. 1, each illustrated gas cylinder could instead be multiple gas cylinders connected to a splitting valve. Each cylinder could also be a connection to a house supply of gas, normally through a wall port in a room in a treatment facility. Also, while not explicitly shown each flow path from a tank through a gas connection 36a-36e may also include an inlet pressure sensor 18a-18e that may be used as described above in connection with FIG. 1. Here, because the flows are connected through t-connections in the conduit to a single pressure regulator 22, some valves 23a-23e may be included before the t-connections so that each flow path could be turned on or off. Otherwise, if one of the tanks was not connected, gas could flow into one of the gas connections 36a-36e, flow through a t-connection, and then flow back out of any of the gas connections 36a-36e that were not connected. Thus, a valve 23a-23e could be electronically controlled such that it would not open unless a pressure was detected by an inlet pressure sensor 18a-18e that may confirm that a gas source is present at the corresponding gas connection 36a-36e. Alternatively, valves 23a-23e may be check valves that operate automatically to close off a flow path when a gas source is not present or is at a low pressure such as due to a malfunction or a cylinder near empty or empty. Note that while t-junctions are illustrated as joining multiple flows together any type of structure to join the flows of multiple conduits together may be used without departing from the scope of the invention. Valves 23a-23e may be located at any point in the flow path between one of the gas connections 36a-36e and the corresponding t-junction.

Each flow path from a tank to the t-junctions may also include a gas heater 20A-20E. In some embodiments, a single heater may be used to heat more than one gas conduit that is fluidly connected to one of the gas cylinders 16a-16e. Of course, a gas heater 20A-20E may be configured in any of the ways discussed in connection with FIG. 1 or otherwise. For example, the heater may heat up the gas connection 36A-36E, be a stand-alone piece of the flow path, or heat the gas within pressure regulator 22. If gas was heated within the pressure regulator 22, then most likely a single heater 20 would be employed but multiple heaters could be used if desired. All of the options discussed in connection with FIG. 1 can be used for the gas connections 36A-36E in FIG. 3.

In this embodiment, the gas flow from multiple cylinders is combined into a flow for output to a single trocar 14 (or other gas delivery device as discussed above). Of course, multiple trocars may be connected to the output channel by replacing tubing 42 with a multi-path tubing set such as multi-patch tubing set 66 illustrated and described in connection with FIG. 11. All of the trocar and other gas delivery device options discussed above in connection with FIG. 1 may be used in connection with insufflator 46 of FIG. 3—any such device may take the place of trocar 14. Flow regulation unit 24 is used to regulate the pressure and flow of insufflation gas through tubing 42 to trocar 14. Any type of flow regulation unit 24 may be used that seeks to maintain a relatively constant pressure in the body cavity, for example by using feedback control in response to one or more pressure sensors as discussed above.

As illustrated, the flow from multiple gas cylinders 16a-16e (which could be 2 or any other number of cylinders) is combined using conduits with T-junctions to join the flows together into a single flow into pressure regulator 22. This embodiment reduces the risk of freeze-up by dividing the high gas flow demands among multiple cylinders. Where two cylinders (or other gas supplies) are used, approximately half of the gas flow will come from each cylinder, thus reducing cooling in each cylinder due to reduced gas flow demands. Where more cylinders are used, the fraction of the gas supplied by each will in most cases be lower.

An input pressure sensor 18a-18e in each flow path may alert insufflator 46 when one or more gas cylinders 16a-16e are not connected to the insufflator. When insufflator 46 detects a missing gas cylinder (or other gas supply), it may close a valve 23a-23e to prevent gas that is flowing into insufflator 46 from one of the gas cylinders 16a-16e to be discharged out of one of the gas connections 36a-36e where no gas cylinder is connected.

In operation, two or more gas cylinders, 16a-16e are connected to one of the gas connections 36a-36e (one for each cylinder) for insufflator 46. When the insufflator is in operation, each cylinder 16a-16e has its own flow channel up to a T-junction, after which the flows are combined to flow into pressure regulator 22. The gas in each flow path may be heated by at least one of the gas heaters 20A-20E. The flow from the cylinders may be combined into a single flow that runs through a conduit into pressure regulator 22 and subsequently flow regulation unit 24. Flow regulation unit 24 regulates the flow characteristics of the gas so as to maintain a relatively constant pressure within a body cavity into which trocar 14 is inserted (e.g. the peritoneal cavity). Flow regulation unit 24 may do so using feedback control that obtains inputs from one or more pressure sensors indicating the pressure inside the body cavity or a pressure that is proportional to that pressure. Flow regulation unit 24 may then regulate gas flow and/or vent pressure to maintain pressure inside the body cavity at a set point. Again, all of the trocar options (or other gas delivery device options) set forth in connection with FIG. 1 may be used for trocar 14 and the techniques for using such trocars and operating insufflator 46 discussed in connection with FIG. 1 may also be used.

In some embodiments, the flow path between gas connections 36a-36e and the t-junctions connecting the flows from the multiple cylinders 16a-16e may contain one way valves 23a-23e to prevent flows from higher pressure cylinders from backflowing into cylinders with low pressures (or into a house supply).

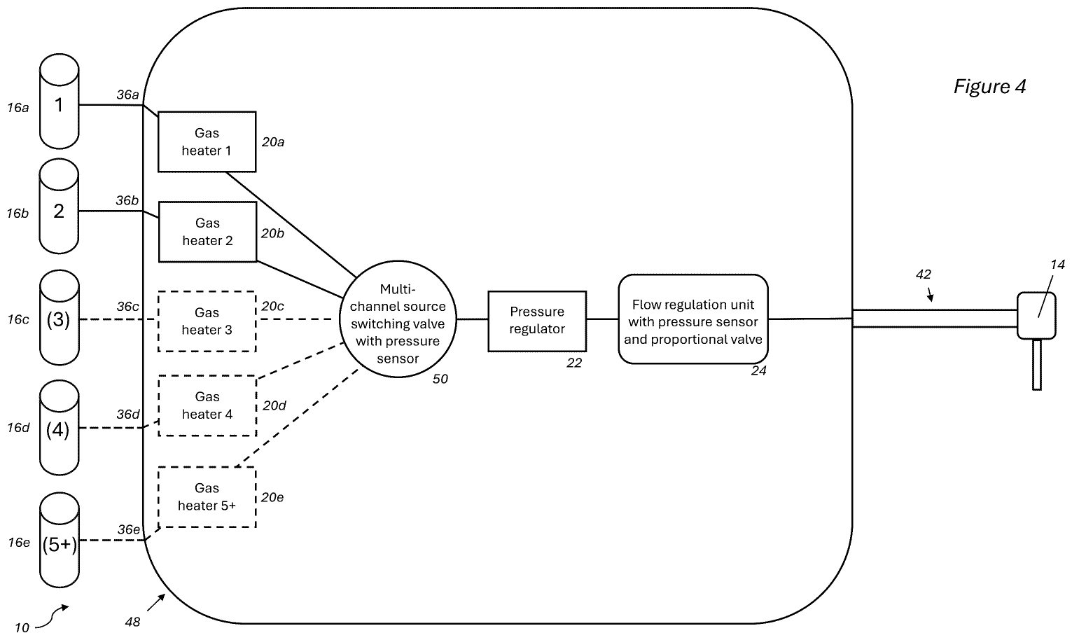

FIG. 4 illustrates another example insufflator 48 that can be used to provide high gas flows while reducing or eliminating the risk of freeze-up. FIG. 4 is similar to FIG. 3 and all of the options discussed for various components of FIG. 3 are applicable to FIG. 4 as well. The main difference between the FIG. 4 and FIG. 3 embodiments is that instead of the flows from the gas cylinders 16a-16e being joined by t-junctions joining multiple conduits together, here a multi-source switching valve 50 is used instead of the t-junctions. Multi-source switching valve may have two or more input conduits and may or may not contain pressure sensors (which are present in this embodiment). A flow path from one of the gas cylinders 16a-16e is connected to one of the inputs of multichannel source switching valve 50. Each flow path from a gas cylinder to multichannel source switching valve may have an input pressure sensor 18 (not explicitly shown) to determine whether a gas cylinder is connected to one of the gas connections 36a-36e. That pressure sensor can be used for other purposes as discussed in connection with FIG. 1. If no cylinder or a malfunctioning or nearly empty cylinder is detected by that pressure sensor, then the multi-channel source switching valve 50 for that flow channel will remain closed. In the illustrated embodiment, each input conduit into multi-channel source switching valve 50 has a pressure sensor built in so that separate input pressure sensors 18 may not be needed. When the sensor inside an input conduit into multi-channel source switching valve 50 detects that a gas cylinder 16a-16e is present, the valve for that conduit may be opened to combine the flows from that cylinder and others that are present into a single flow into pressure regulator 22.

FIG. 4, however, can be used in a different mode of operation. Instead of combining the flow from multiple cylinders, the embodiment of FIG. 4 could instead cause gas from one of the cylinders 16a-16e to flow for a short time (10 seconds-two minutes) and then switch the flow to a different gas cylinder 16a-16e. If there are more than two cylinders (or other gas sources) present, then the flow can be switched to each cylinder in a sequence. In such an embodiment, while high gas flows would be demanded from a single cylinder while it is the active cylinder, they would only be demanded for a short time, thus allowing the cylinder time to warm up again somewhat before gas flows again. This round robin use of two or more cylinders 16a-16e (two to five cylinders in this embodiment), could be accomplished by opening a single conduit in multichannel source switching valve 50 at a time. Note that as per all of the embodiments disclosed in this patent, each cylinder 16a-16e could be a house gas connection (e.g. through a port in the wall of a room in a medical facility) or could be multiple cylinders joined with splitting valve.

In some embodiments multichannel source switching valve 50 has a separate valve that can open and close each conduit individually. In other embodiments, multichannel source switching valve may open and close a flow path from each input conduit to the output conduit simultaneously. In such an embodiment, one may need additional valves to prevent backflow out of gas connections 36a-36e if a cylinder is not connected. Alternatively, in such an embodiment, multichannel source switching valve 50 could be controlled such that it only allows flow if all cylinders are connected.

In operation, two or more gas cylinders (or a house gas supply or a mixture of cylinders and a house gas supply connections), 16a-16e are connected to one of the gas connections 36a-36e (one for each cylinder) for insufflator 48. When the insufflator is in operation, each cylinder 16a-16e has its own flow channel with a heater 20a-20e up to multichannel source switching valve 50, after which two or more of the flows may be combined to flow into pressure regulator 22. The flows are combined by the multichannel source switching valve that is capable of combining flows from multiple inputs into a single output flow. Pressure regulator 22 then controls the pressure of the flow while flow regulation unit regulates gas flow based upon sensed pressure to maintain as stable as possible a pressure in the body cavity based upon a desired pressure setting set by a user. Feedback control using one or more pressure sensors may be used to regulate the pressure as discussed above. The user may set a desired pressure in the body cavity using user interface 40 (not explicitly shown). Flow regulation unit 24 seeks to control the flow of gas and/or venting of pressure in order to keep the pressure close to the set point.

The gas in each flow path is heated by at least one of the gas heaters 20A-20E. The flow from the cylinders (or house supply) may be combined into a single flow that runs through a conduit into pressure regulator 22 and subsequently flow regulation unit 24. Flow regulation unit 24 regulates the flow characteristics of the gas so as to maintain a relatively constant pressure within a body cavity into which trocar 14 (or other gas delivery device) is inserted (e.g. the peritoneal cavity). Again, all of the trocar (or other gas delivery device) options set forth in connection with FIG. 1 may be used for trocar 14 and the techniques for using such trocars (or other gas delivery devices) and operating insufflator 48 discussed in connection with FIG. 1 may also be used. In addition, a needle or other device used to deliver gas to a body cavity can be used in place of trocar 14. The trocar 14 (or other device) is inserted into the body cavity and delivers the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 (not explicitly shown) of insufflator 48 may control the gas from each cylinder (or house supply) to flow in a round robin fashion. Instead of combining the flow from multiple cylinders, the embodiment of FIG. 4 could instead be operated by the controller to cause gas to flow from one of the cylinders 16a-16e for a short time (10 seconds-two minutes) and then switch the flow to a different gas cylinder 16a-16e. Flow may come from all available cylinders with only one cylinder operating at a time in a sequence. Freeze-up may be deterred by flowing a high amount of gas from a single supply for only a short time period. As discussed above, each illustrated cylinder 16 may be a plurality of cylinders joined by a splitting valve.

In some embodiments, rather than using a single trocar 14, multiple trocars (or other gas delivery devices) can be connected to the output of insufflator 48 using a multipath tubing set such as, for example multipath tubing set 66 described below in connection with FIG. 11.

Controller 38 can control multichannel source switching valve 50 such that input channels of valve 50 are not connected to the output of valve 50 when the input is not connected to a gas source or is connected to a gas source that is malfunctioning or is almost empty. In some embodiments, multi-channel source switching valve 50 may have pressure sensors on each input so that controller 38 may easily determine whether (a) a cylinder or house supply of gas or nothing is connected to each particular gas connection 36a-36e of insufflator 48, (b) a cylinder or house supply is malfunctioning as indicated by a low pressure condition, and/or (c) a cylinder is getting low on gas as indicated by declining pressure. In some embodiments, each gas input to the insufflator may pass through an inlet pressure sensor 18a-18e (not explicitly shown) to make these same determinations. Controller 38 may then control multi-channel source switching valve 50 so as to not connect any input to its output where no gas source is connected or where the gas source is malfunctioning or nearly depleted.

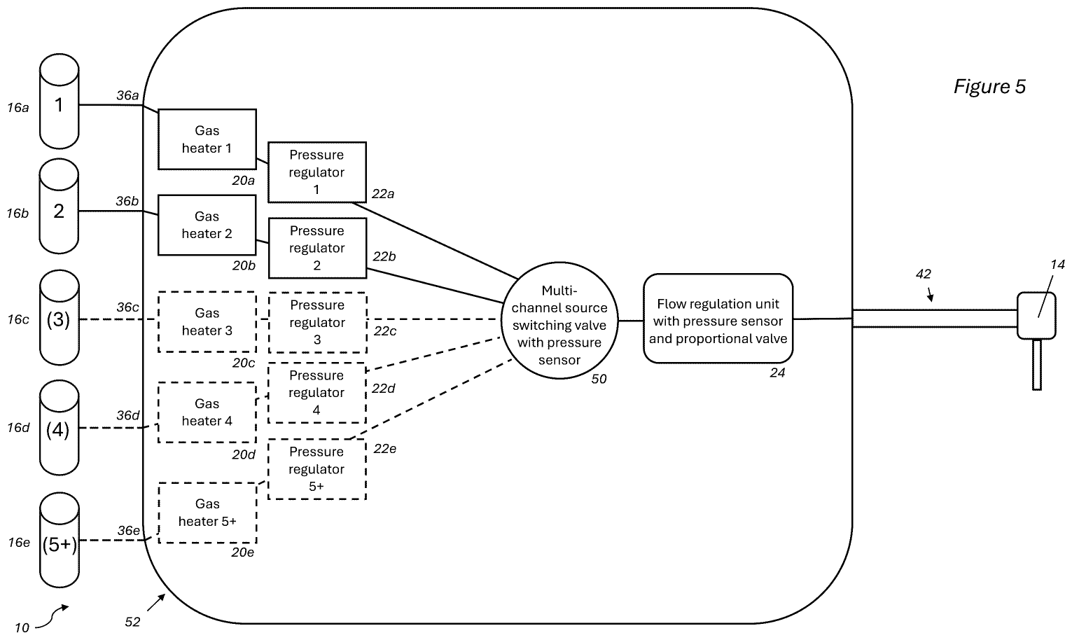

FIG. 5 illustrates another example insufflator 52 that can be used to provide high gas flows while reducing or eliminating the risk of freeze-up. FIG. 5 is similar to FIG. 2 and all of the options discussed for various components of FIG. 2 are applicable to FIG. 5 as well. The main difference between the FIG. 5 and FIG. 2 embodiments is that instead of the flows from the pressure regulators 22a-22e being joined by t-junctions joining multiple conduits together, here a multi-source switching valve 50 with pressure sensors is used instead of the t-junctions. The pressure sensors may be omitted from the multi-source switching valve 50 without departing from the scope of the invention. Multi-source switching valve 50 may have two or more input conduits, typically one for each cylinder or flow path that it may join the flows of together. Note that obviously the flows of two or more gas cylinders could be joined by a t-junction (or other method of joining flows from multiple conduits) and the combined flow could be provided to an input of multichannel source switching valve 50. In the illustrated embodiment, a flow path from one of the gas cylinders 16a-16e is connected to one of the inputs of multichannel source switching valve 50 through one of the pressure regulators 22a-22e. Each flow path from a gas cylinder to multichannel source switching valve may have an input pressure sensor 18 to determine whether a gas cylinder is connected to one of the gas connections 36a-36e as such as was discussed in connection with FIG. 1 and FIG. 4. That pressure sensor can be used for other purposes as discussed in connection with FIG. 1. If no cylinder or a malfunctioning or nearly empty cylinder is detected by that pressure sensor, then the multi-channel source switching valve 50 will keep that flow channel closed. In the illustrated embodiment, each input conduit into multi-channel source switching valve 50 has a pressure sensor built in so that separate input pressure sensors 18 may not be needed. When the sensor inside a conduit into multi-channel source switching valve 50 detects that a gas cylinder 16a-16e is present, the valve inside multi-channel source switching valve 50 for that conduit may be opened to combine the flows from that cylinder and others that are present into a single flow into flow regulation unit 24.

FIG. 5, like the one in FIG. 4, can also be used in a different mode of operation. Instead of combining the flow from multiple cylinders into a single flow, the embodiment of FIG. 5 could instead cause gas to flow from one of the cylinders 16a-16e for a short time (10 seconds-two minutes) and then switch the flow to a different gas cylinder 16a-16e to flow for a second short time (which could be the same or different as the first time). This round-robin flow can be continued for each cylinder present in a sequence. In such an embodiment, while high gas flows would be demanded from a single cylinder while the valve for that cylinder was open, they would only be demanded for a short time, thus allowing the cylinder time to warm up again somewhat before gas flows f particular cylinder again. This round robin use of two or more cylinders 16a-16e (two to five cylinders in this embodiment but it could be any number of cylinders of 2 or more), could be accomplished by opening a single conduit in multichannel source switching valve 50 at a time. Controller 38 would close that valve when opening the valve for the next cylinder. Note that as per all of the embodiments disclosed in this patent, each cylinder 16a-16e could be a house gas connection (e.g. through a port in the wall of a room in a medical facility) or could be multiple cylinders joined with splitting valve.

In some embodiments multichannel source switching valve 50 has a separate valve that can open and close each conduit individually. In other embodiments, multichannel source switching valve may open and close a flow path from each input conduit to the output conduit simultaneously. In such an embodiment, one may need additional valves to prevent backflow out of gas connections 36a-36e if a cylinder is not connected. Alternatively, in such an embodiment, multichannel source switching valve 50 could be controlled such that it only allows flow if all cylinders are connected.

In operation, two or more gas cylinders (or a house gas supply or a mixture of cylinders and a house gas supply connections), 16a-16e are connected to one of the gas connections 36a-36e (one for each cylinder) for insufflator 52. When the insufflator 52 is in operation, each cylinder 16a-16e has its own flow channel with a heater 20a-20e and pressure regulator 22a-22e up to multichannel source switching valve 50, after which two or more of the flows may be combined to flow into flow regulation unit 24. The flows are combined by the multichannel source switching valve that is capable of combining flows from multiple inputs into a single output flow. Flow regulation unit 24 regulates gas flow based upon sensed pressure to maintain as stable as possible a pressure in the body cavity based upon a desired pressure setting set by a user. The user may set a desired pressure in the body cavity using user interface 40 (not explicitly shown). Pressure may be sensed using one or more pressure sensors in insufflator 52, in tubing 42, and/or in or on trocar 14 (or another gas delivery device). In some embodiments, pressure may be sensed both in or on trocar 14 (or some other device inserted into the body cavity) and in insufflator 52 to have redundant pressure sensing. Redundant sensors can also be within insufflator 52. Redundant sensors could be used to detect a malfunction of one of the sensors.

The gas in each flow path may be heated by at least one of the gas heaters 20A-20E and then may be pressure regulated by at least one of the pressure regulators 22a-22e. The flow from the cylinders (or house supply) may be combined into a single flow that runs through a conduit into flow regulation unit 24. Flow regulation unit 24 regulates the flow characteristics of the gas so as to maintain a relatively constant pressure within a body cavity into which trocar 14 is inserted (e.g. the peritoneal cavity). It may do so using feedback control as described above using one or more pressure sensors to sense the pressure in the body cavity or a proportional pressure to that one. Again, all of the trocar (or other gas delivery device) options set forth in connection with FIG. 1 may be used for trocar 14 and the techniques for using such trocars (or other gas delivery devices) and operating insufflator 52 discussed in connection with FIG. 1 may also be used. In addition, a needle or other device used to deliver gas to a body cavity can be used in place of trocar 14. The trocar 14 (or other device) is inserted into the body cavity and delivers the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 (not explicitly shown) of insufflator 52 may control the gas from each cylinder (or house supply) to flow in a round robin fashion. Instead of combining the flow from multiple cylinders, the embodiment of FIG. 5 could instead be operated by the controller 38 to cause gas to flow from one of the cylinders 16a-16e for a short time (10 seconds-two minutes) and then switch the flow to a different gas cylinder 16a-16e to flow for a second short time (which could be the same or different as the first short time). This process may continue with a third cylinder and/or additional cylinders that are connected. Freeze-up may be deterred by flowing gas at a high flow rate from a single gas supply for only a short time period. Flow can be in a round robin fashion from each functional gas supply in a sequence. As discussed above, each illustrated cylinder 16 may be a house supply or may be a plurality of cylinders joined by a splitting valve.

In some embodiments, rather than using a single trocar 14, multiple trocars can be connected to the output of insufflator 52 using a multipath tubing set such as, for example multipath tubing set 66 described below in connection with FIG. 11.

Controller 38 can control multichannel source switching valve 50 such that input channels of valve 50 are not connected to the output of valve 50 when the input is not connected to a gas source or is connected to a gas source that is malfunctioning or is almost empty. In some embodiments, multichannel source switching valve 50 may have pressure sensors on each input so that controller 38 may easily determine whether (a) a cylinder or house supply of gas or nothing is connected to each particular gas connection 36a-36e of insufflator 52, (b) a cylinder or house supply is malfunctioning as indicated by a low pressure condition, and/or (c) a cylinder is getting low on gas as indicated by declining pressure. In some embodiments, each gas input to the insufflator may pass through an inlet pressure sensor 18a-18e (not explicitly shown) to make these same determinations. Controller 38 may then control multi-channel source switching valve 50 so as to not connect any particular input to the output of valve 50 where no gas source is connected to that particular input or where the gas source is malfunctioning or nearly depleted.

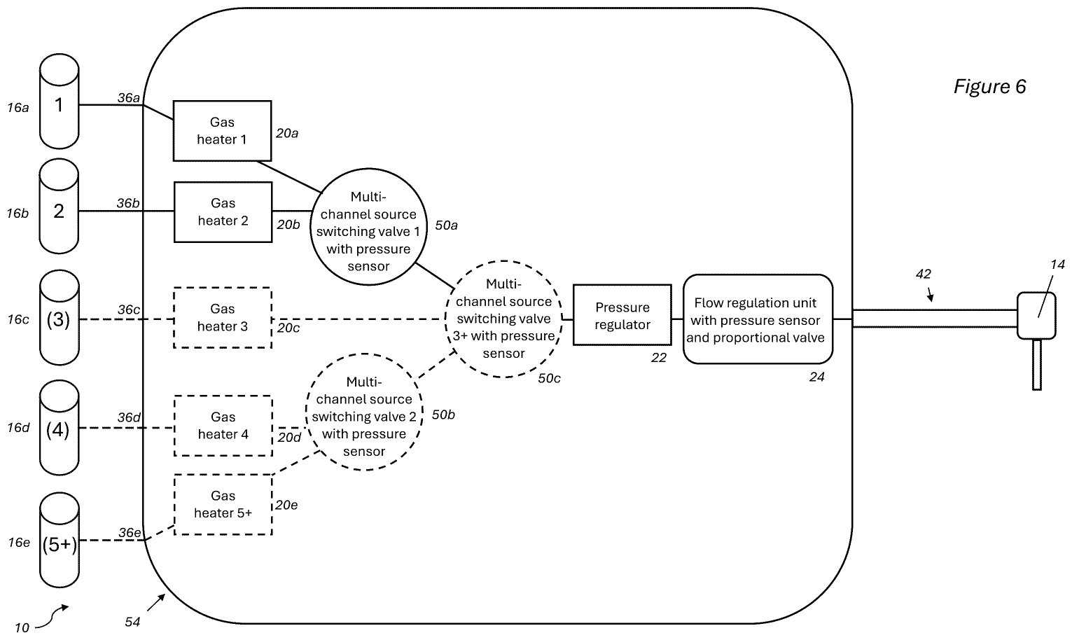

FIG. 6 illustrates another example insufflator 54 that can be used to provide high gas flows while reducing or eliminating the risk of freeze-up. FIG. 6 is similar to FIG. 4 and all of the options discussed for various components of FIG. 4 are applicable to FIG. 6 as well. The main difference between the FIG. 6 and FIG. 4 embodiments is that instead of the flows from the gas cylinders 16a-16e being joined by a single multi-source switching valve 50, multiple multi-channel source switching valves 50a-50c arranged in a tree structure may be used instead. Multi-channel source switching valves 50a-50c may have two or more input conduits. However, the more inputs a multi-source switching valve 50a-50c has, the more complex it is to make and the more expensive it is. The embodiment of FIG. 6 may use two or three input multi-channel source switching valves 50a-50c arranged in a tree structure to potentially reduce costs as compared to the use of a multi-channel source switching valve 50 with many inputs.

A flow path from one of the gas cylinders 16a-16e is connected to one of the inputs of multichannel source switching valves 50a, 50b or 50c. Each flow path from a gas cylinder to one of the multichannel source switching valves 50a, 50b, or 50c may have an input pressure sensor 18 to determine whether a gas cylinder is connected to one of the gas connections 36a-36e. That pressure sensor can be used for other purposes as discussed in connection with FIG. 1. If no cylinder or a malfunctioning or nearly empty cylinder is detected by that pressure sensor, then the multi-channel source switching valve 50a 50b, or 50c for that flow channel will remain closed. In the illustrated embodiment, each input conduit into one of the multi-channel source switching valves 50a-50c has a pressure sensor built in so that separate input pressure sensors 18 may not be needed. When the sensor inside a conduit into one of the multi-channel source switching valves 50a-50c detects that a gas cylinder 16a-16e is present, the valve for that conduit may be opened to combine the flows from that cylinder and others that are present into a single flow into pressure regulator 22.

The multi-channel source switching valves 50a-50c can be controlled to connect one or more cylinders to the output of multi-source switching valve 50c. In this embodiment, the insufflator 54 can operate with a single cylinder connected or two or more cylinders connected. The valves on multi-source switching valve 50a-50c simply need to be set to open valves where an operational cylinder (or other gas supply) 16a-16e is present and to close valves where 10 no operational cylinder 16a-16e (or other gas supply) is present.

FIG. 6, however, can be used in a different mode of operation like prior embodiments having a multi-source switching valve 50. Instead of combining the flow from multiple cylinders, the embodiment of FIG. 6 could instead cause gas to flow from one of the cylinders 16a-16e to flow for a short time (10 seconds-two minutes) and then switch the flow to a different gas cylinder 16a-16e to flow for a second short time (which may or may not be the same as the first time), and so on for the number of cylinders connected to insufflator 54. In such an embodiment, while high gas flows would be demanded from a single cylinder, they would only be demanded for a short time, thus allowing the cylinder time to warm up again somewhat before gas flows again from that particular cylinder. This round robin use of two or more cylinders 16a-16e (two to five cylinders in this embodiment), could be accomplished by opening the appropriate single conduit in one of the multi-channel source switching valves 50a-50c at a time. In this embodiment, the appropriate valve in multi-source switching valve 50c would also need to be opened for a cylinder feeding the tree structure before that valve. Note that as per all of the embodiments disclosed in this patent, each cylinder 16a-16e could be a house gas connection (e.g. through a port in the wall of a room in a medical facility) or could be multiple cylinders joined with splitting valve.

In some embodiments multichannel source switching valve 50 has a separate valve that can open and close each conduit individually. In other embodiments, multichannel source switching valve may open and close a flow path from each input conduit to the output conduit simultaneously. In such an embodiment, one may need additional valves to prevent backflow out of gas connections 36a-36e if a cylinder is not connected. Alternatively, in such an embodiment, multichannel source switching valve 50 could be controlled such that it only allows flow if all cylinders are connected.

In operation, two or more gas cylinders (or a house gas supply or a mixture of cylinders and a house gas supply connections), 16a-16e are connected to one of the gas connections 36a-36e (one for each cylinder or house supply connection) for insufflator 54. When the insufflator is in operation, each cylinder 16a-16e has its own flow channel up to multichannel source switching valves 50a-50c, after which two or more of the flows may be combined to flow into pressure regulator 22 and flow regulation unit 24. The flows are combined by the one or more of the multichannel source switching valves 50a-50c that are capable of combining flows from multiple inputs into a single output flow. Flow regulation unit 24 regulates gas flow based upon sensed pressure to maintain a pressure in the body cavity based upon a desired pressure setting set by a user. It may use feedback control as discussed above using one or more pressure sensors that sense the pressure inside the body cavity or a proportional pressure. Controller 38 (not explicitly shown) controls the flow of insufflation gas from flow regulation unit 24 to attempt to maintain pressure at a pressure set-point. The user may set a desired pressure in the body cavity using user interface 40 (not explicitly shown). Pressure may be sensed using one or more pressure sensors in insufflator 54, in tubing 42, and/or in or on trocar 14. In some embodiments, pressure may be sensed both in or on trocar 14 (or some other device inserted into the body cavity) and in insufflator 54 to have redundant pressure sensing. Redundant sensors can also be within insufflator 54. Redundant sensors could be used to detect a malfunction of one of the sensors.

The gas in each input flow path is heated by at least one of the gas heaters 20A-20E. The flow from the cylinders (or house supply) may be combined into a single flow that runs through a conduit into pressure regulator 22 and then flow regulation unit 24. Flow regulation unit 24 regulates the flow characteristics of the gas so as to maintain a relatively constant pressure within a body cavity into which trocar 14 (or another gas delivery device such as a needle) is inserted (e.g. the peritoneal cavity). Again, all of the trocar (or other gas delivery device) options set forth in connection with FIG. 1 may be used for trocar 14 and the techniques for using such trocars (and other devices) and operating insufflator 54 discussed in connection with FIG. 1 may also be used. In addition, a needle or other device used to deliver gas to a body cavity can be used in place of trocar 14. The trocar 14 (or other device) is inserted into the body cavity and delivers the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 (not explicitly shown) of insufflator 54 may control the gas from each cylinder (or house supply) to flow in a round robin fashion. Instead of combining the flow from multiple cylinders, the embodiment of FIG. 6 could instead be operated by the controller 38 to cause gas to flow from one of the cylinders (or other gas sources) 16a-16e for a first period of time (10 seconds-two minutes) and then switch the flow to a different gas cylinder 16a-16e for a second period of time (which may be the same or different than the first period of time) and so on for each cylinder connected. This can be done with all of the cylinders (or other gas sources) that are connected to the insufflator. Freeze-up may be deterred by flowing gas at a high flow rate from a single gas supply for only a short time period and then switching to a different gas supply. As discussed above, each illustrated cylinder 16 may be a plurality of cylinders joined by a splitting valve.

In some embodiments, rather than using a single trocar 14, multiple trocars can be connected to the output of insufflator 54 using a multipath tubing set such as, for example multipath tubing set 66 described below in connection with FIG. 11.

Controller 38 control can multichannel source switching valves 50a-50c such that input channels of valves 50a-50c are not connected to the output of the valve in question when the input is not connected to a gas source or is connected to a gas source that is malfunctioning or is almost empty. In some embodiments, multi-channel source switching valves 50a-50c may have pressure sensors on each input so that controller 38 may easily determine whether (a) a cylinder or house supply of gas or nothing is connected to each particular gas connection 36a-36e of insufflator 54, (b) cylinder or house supply is malfunctioning as indicated by a low pressure condition, and/or (c) a cylinder is getting low on gas as indicated by declining pressure. In some embodiments, each gas input to the insufflator may pass through an inlet pressure sensor 18a-18e (not explicitly shown) to make these same determinations. Controller 38 may then control multi-channel source switching valves 50a-50c so as to not connect any particular input to the output of any of those valves where no gas source is connected to that particular input or where the gas source is malfunctioning or nearly depleted.

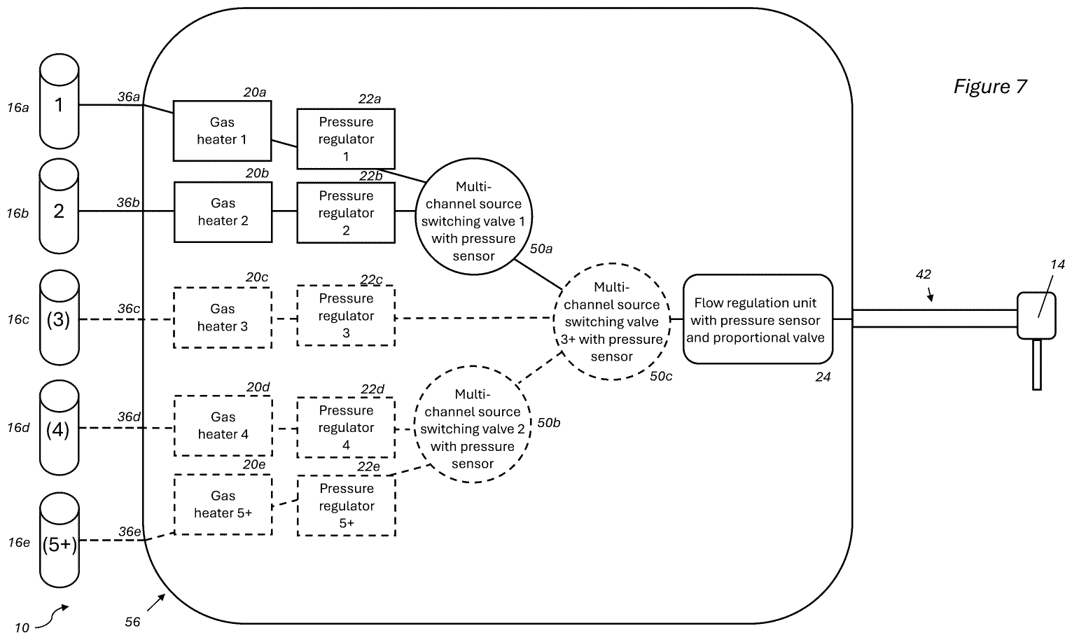

FIG. 7 illustrates another example insufflator 56 that can be used to provide high gas flows while reducing or eliminating the risk of freeze-up. FIG. 7 is similar to FIG. 5 (as well as FIG. 2) and all of the options discussed for various components of FIG. 5 (as well as FIG. 2) are applicable to FIG. 7 as well. The main difference between the FIG. 7 and FIG. 5 embodiments is that instead of the flows from the gas cylinders 16a-16e being joined by a single multi-source switching valve 50, multiple multi-channel source switching valves 50a-50c arranged in a tree structure are used instead. Multi-channel source switching valves 50a-50c may have two or more input conduits. However, the more inputs a multi-source switching valve 50a-50c has, the more complex it is to make and the more expensive it is. The embodiment of FIG. 7 may use two or three input multi-channel source switching valves 50a-50c arranged in a tree structure to potentially reduce costs as compared to the use of a multi-channel source switching valve 50 with many inputs.

A flow path from one of the gas cylinders 16a-16e (or other gas sources) is connected to one of the inputs of multichannel source switching valves 50a, 50b or 50c. Each flow path from a gas cylinder (or other gas source) to one of the multichannel source switching valves 50a, 50b, or 50c may have an input pressure sensor 18 to determine whether a gas cylinder is connected to one of the gas connections 36a-36e. That pressure sensor can be used for other purposes as discussed in connection with FIG. 1. If no cylinder or a malfunctioning or nearly empty cylinder is detected by that pressure sensor, then the multi-channel source switching valve 50a 50b, or 50c for that flow channel will remain closed. In the illustrated embodiment, each input conduit into one of the multi-channel source switching valves 50a-50c has a pressure sensor built in so that separate input pressure sensors 18 may not be needed. When the sensor inside a conduit into one of the multichannel source switching valves 50a-50c detects that a gas cylinder 16a-16e is present, the valve for that conduit may be opened to combine the flows from that cylinder and others that are present into a single flow into flow regulation unit 24.

The multi-channel source switching valves 50a-50c can be controlled to connect one or more cylinders to the output of multi-source switching valve 50c. In this embodiment, the insufflator 56 can operate with a single cylinder connected or two or more cylinders connected. The valves on multi-source switching valve 50a-50c simply should be set to open valves where an operational cylinder 16a-16e is present and to close valves where no operational cylinder 16a-16e is present.

FIG. 7, however, can be used in a different mode of operation like prior embodiments having a multi-source switching valve 50. Instead of combining the flow from multiple cylinders, the embodiment of FIG. 7 could instead cause gas to flow from one of the cylinders 16a-16e to flow for a short time (10 seconds-two minutes) and then switch the flow to a different gas cylinder 16a-16e for a second short time (which may or may not be the same as the first short time), and so on for the number of cylinders connected to insufflator 56. In such an embodiment, while high gas flows would be demanded from a single cylinder, they would only be demanded for a short time, thus allowing the cylinder time to warm up again somewhat before gas flows again from that particular cylinder. This round robin use of two or more cylinders 16a-16e (two to five cylinders in this embodiment), could be accomplished by opening the appropriate single conduit in one of the multi-channel source switching valves 50a-50c at a time. In this embodiment, the appropriate valve in multi-source switching valve 50c would also need to be opened for a cylinder feeding the tree structure before that valve. Note that as per all of the embodiments disclosed in this patent, each cylinder 16a-16e could be a house gas connection (e.g. through a port in the wall of a room in a medical facility) or could be multiple cylinders joined with splitting valve.

In some embodiments multichannel source switching valve 50a-50c has a separate valve that can open and close each conduit individually. In other embodiments, multichannel source switching valve may open and close a flow path from each input conduit to the output conduit simultaneously. In such an embodiment, one may need additional valves to prevent backflow out of gas connections 36a-36e if a cylinder is not connected. Alternatively, in such an embodiment, multichannel source switching valve 50a-50c could be controlled such that it only allows flow if all cylinders are connected.

In operation, two or more gas cylinders (or a house gas supply or a mixture of cylinders and a house gas supply connections), 16a-16e are connected to one of the gas connections 36a-36e (one for each cylinder or house supply connection) for insufflator 56. When the insufflator is in operation, each cylinder 16a-16e has its own flow channel up to multichannel source switching valves 50a-50c, after which two or more of the flows may be combined to flow into flow regulation unit 24. The flows are combined by one or more of the multichannel source switching valves 50a-50c that are capable of combining flows from multiple inputs into a single output flow. Flow regulation unit 24 regulates gas flow based upon sensed pressure to maintain a relatively stable pressure in the body cavity based upon a desired pressure setpoint that is set by a user. As discussed above, it does so via feedback control in response to one or more pressure sensors. Controller 38 (not explicitly shown) controls the flow of insufflation gas from flow regulation unit 24 to attempt to maintain pressure at a pressure set-point. The user may set a desired pressure in the body cavity using user interface 40 (not explicitly shown). Pressure may be sensed using one or more pressure sensors in insufflator 56, in tubing 42, and/or in or on trocar 14. In some embodiments, pressure may be sensed both in or on trocar 14 (or some other device inserted into the body cavity) and in insufflator 56 to have redundant pressure sensing. Redundant sensors can also be within insufflator 56. Redundant sensors could be used to detect a malfunction of one of the sensors.

The gas in each input flow path is heated by at least one of the gas heaters 20A-20E and regulated by one of the pressure regulators 22a-22e. The flow from the cylinders (or house supply) may be combined into a single flow that runs through a conduit into flow regulation unit 24. Flow regulation unit 24 regulates the flow characteristics of the gas so as to maintain a relatively constant pressure within a body cavity into which trocar 14 (or another gas delivery device such as a needle) is inserted (e.g. the peritoneal cavity). Again, all of the trocar (or other gas delivery device) options set forth in connection with FIG. 1 may be used for trocar 14 and the techniques for using such trocars (or other gas delivery devices) and operating insufflator 56 discussed in connection with FIG. 1 may also be used. In addition, a needle or other device used to deliver gas to a body cavity can be used in place of trocar 14. The trocar 14 (or other device) is inserted into the body cavity and delivers the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 (not explicitly shown) of insufflator 56 may control the gas from each cylinder (or house supply) to flow in a round robin fashion. Instead of combining the flow from multiple cylinders, the embodiment of FIG. 7 could instead be operated by the controller 38 to cause gas to flow from one of the cylinders (or other gas sources) 16a-16e for a first period of time (10 seconds-two minutes) and then switch the flow to a different gas cylinder 16a-16e for a second period of time (which may be the same or different than the first period of time). This can be done with how many ever cylinders (or other gas sources) are connected to the insufflator. Freeze-up may be deterred by flowing gas at a high flow rate from a single gas supply for only a short time period and then switching to a different gas supply. As discussed above, each illustrated cylinder 16 may be a plurality of cylinders joined by a splitting valve.

Another alternative would be for multichannel source switching valve 50A to alternate between cylinders 16a and 16b while multichannel source switching valve 50c alternates between cylinders 16d and 16e. This would allow rotation between cylinders to deter freeze-up but would allow the flow of multiple cylinders to still be combined. Controller 38 could also be programmed to alternate flow between the output of multichannel source switching valve 50a and multichannel source switching valve 50b and the input coupled to cylinder 16c.

In some embodiments, rather than using a single trocar 14, multiple trocars (or other gas delivery devices) can be connected to the output of insufflator 56 using a multipath tubing set such as, for example multipath tubing set 66 described below in connection with FIG. 11.

Controller 38 can control multichannel source switching valves 50a-50c such that input channels of valves 50a-50c are not connected to the output of the valve in question when the input is not connected to a gas source or is connected to a gas source that is malfunctioning or is almost empty. In some embodiments, multi-channel source switching valves 50a-50c may have pressure sensors on each input so that controller 38 may easily determine whether (a) a cylinder or house supply of gas or nothing is connected to each particular gas connection 36a-36e of insufflator 56, (b) a cylinder or house supply is malfunctioning as indicated by a low pressure condition, and/or (c) a cylinder is getting low on gas as indicated by declining pressure. In some embodiments, each gas input to the insufflator may pass through an inlet pressure sensor 18a-18e (not explicitly shown) to make these same determinations. Controller 38 may then control multi-channel source switching valves 50a-50c so as to not connect any particular input to the output of any of those valves where no gas source is connected to that particular input or where the gas source is malfunctioning or nearly depleted.

Insufflator 56 thus supplies insufflation gas to a body cavity and controls the flow such that insufflator 56 seeks to maintain the pressure of the body cavity at a pressure set point input by the user.

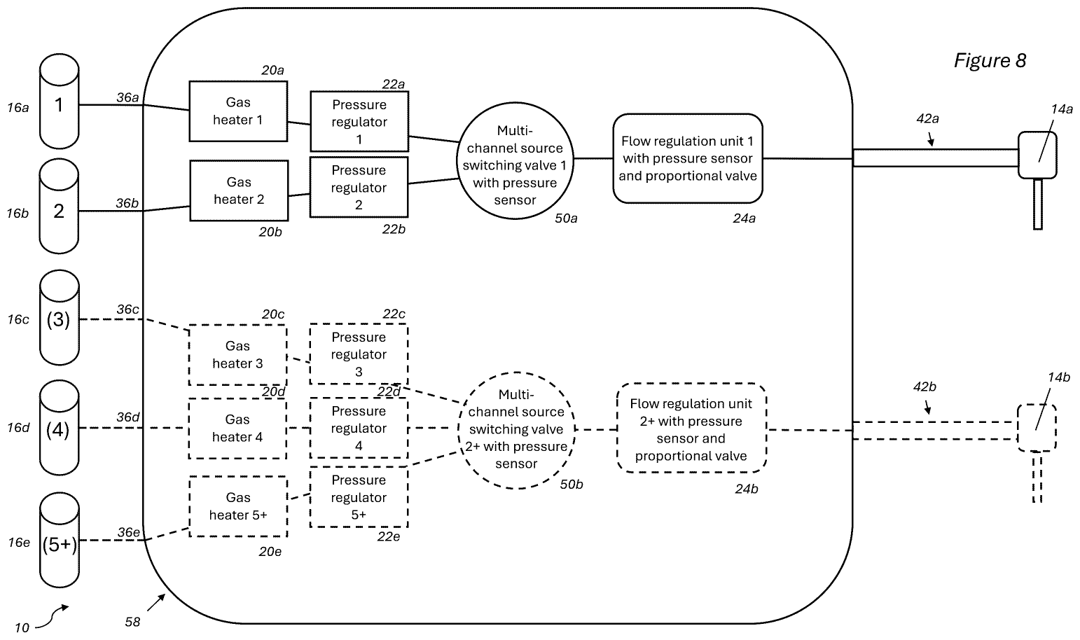

FIG. 8 illustrates another example insufflator 58 that can be used to provide high gas flows while reducing or eliminating the risk of freeze-up. FIG. 8 is similar to FIG. 5 (as well as FIG. 2) and all of the options discussed for various components of FIG. 5 (as well as FIG. 2) are applicable to FIG. 8 as well. The main difference between the FIG. 8 and FIG. 5 embodiments is that FIG. 8 illustrates a dual output port insufflator that can provide gas through separate outputs to separate trocars 14a and 14b. While this embodiment has two outputs, insufflator 58 could have any number of outputs without departing from the scope of the invention. Each output could be connected to (a) a single tank input and have the structure illustrated in FIG. 1, (b) a double tank input and have the structure illustrated at the top of FIG. 8, or (c) three or more tank inputs and have the structure illustrated at the bottom of FIG. 8. Each output could also be connected to the structures feeding any single trocar in FIG. 2-7 or 10.

The trend in minimally invasive surgery is to use trocars with smaller diameters to reduce incision sizes for the patient. Smaller incision sizes have multiple benefits for the patient, such as faster healing time. Smaller trocars, however, limit gas flow to the patient and have an upper limit of the number of liters per minute of gas that can flow through the trocar. Thus, even if an insufflator is capable of supplying 30-60 liters per minute of gas, a given trocar may only allow 8-12 lpm of gas flow if it has a smaller diameter. Obviously, lower diameters may result in even more limited flow.

But using smaller trocars does not eliminate the need for high gas flows, particularly in procedures such as hysterectomies where large leaks are present. High gas flows are also needed, as discussed above, in procedures where a lot of suction and/or smoke evacuation is employed. Because of the physical limits of smaller diameter trocars, gas flow can be provided to multiple trocars in order to provide a cumulative gas flow that is more desirable than the flow possible through a single trocar.

The insufflator 58 of FIG. 8 is particularly useful for use with smaller diameter trocars. Insufflator 58 has multiple outputs that provide gas flow to multiple trocars 14a-14b. Again, while two trocars are shown, insufflator 58 could have 3, 4, 5 or more outputs. One could use or of structure similar to the top bottom channels insufflator 58 for each of the outputs or use structure similar to that shown in FIGS. 2-7 and 10 for one or more of the outputs.

The top output of insufflator 58 is driven by a two cylinder (or other gas source as discussed above) version of the embodiment of FIG. 5, while the bottom output of insufflator 58 is driven by a three cylinder version of the embodiment of FIG. 5. However, each output is not independent. Instead, each channel can be operated under common control of controller 38 (not explicitly shown). Insufflator 58 can advantageously control both outputs so as to maximize gas output and minimize interruption of gas flow. In most conventional insufflators, gas flow ceases temporarily so that a measurement of pressure in the abdomen can be made. The embodiment of FIG. 8 allows insufflation to be ceased by shutting off one of the on/off valves in flow regulation unit 24a or 24b. The other on/off valve in the other flow regulation unit 24a or 24b may remain open and flow can continue while the pressure is being measured, thus reducing the impact of stopping the flow to take a pressure measurement that one would normally see in a conventional insufflator. Also, because pressure measurements can be taken by both flow regulation units 24a and 24b (advantageously at different times), pressure readings can be compared to identify a bad pressure sensor and provide a degree of redundancy. Of course, either trocar 14a, trocar 14b, or both could include a pressure sensor in or on the trocar to facilitate pressure sensing at the patient so that insufflation did not have to be stopped at any time. In such an arrangement, the pressure sensors in flow regulation units 24a, 24b, could be used as backups in case of a malfunction of the pressure sensors in the trocars.

Another advantage of the embodiment illustrated in FIG. 8 is that multiple types of insufflation gas could be provided and blended together with one type of gas going to each trocar 14a and 14b. The insufflator could control the flow to each trocar so as to obtain an approximate percentage of blend of the multiple gases set by the user through the user interface.

If the embodiment of FIG. 8 uses a single gas cylinder for a channel using, for example, the structures illustrated in FIG. 1, then that channel could operate like the embodiment of FIG. 1 except that both outputs to trocars 14a and 14b would be controlled at the same time. If the embodiment of FIG. 8 uses two or more gas cylinders like the options shown at the top or bottom of FIG. 8, then each channel can be operated such that a fraction of the gas is provided by each cylinder simultaneously (e.g. all valves open for the multiple conduits of multi-channel source switching valves 50a or 50b) or each channel could be operated in a round robin fashion as described above in connection with FIGS. 4-7 such that all of the gas for a particular output to one of the trocars 14a or 14b is being provided by a single gas cylinder 16a-16e for a short period of time and then the gas would be provided by a different cylinder for the next short period of time. If a channel uses multiple cylinders, that channel could also switch to single cylinder operation where another cylinder becomes low or or empty malfunctions.

In operation, two or more gas cylinders (or a house gas supply or a mixture of cylinders and a house gas supply connections), 16a-16e are connected to one of the gas connections 36a-36e (one for each cylinder or house supply connection) for insufflator 58. When the insufflator is in operation, each cylinder 16a-16e has its own flow channel up to multichannel source switching valves 50a-50b, after which two or more of the flows may be combined to flow into one of the flow regulation units 24a-24b, as illustrated. Insufflator 58 has two output channels but may have more output channels without departing from the scope of the invention. The flows for each output channel may be combined by one or more of the multichannel source switching valves 50a-50b that are capable of combining flows from multiple inputs into a single output flow. Flow regulation units 24a-24b may regulate gas flow based upon sensed pressure to maintain a relatively stable pressure in the body cavity based upon a desired pressure setting set by a user. Controller (not explicitly shown) controls the flow of insufflation gas from flow regulation units 24a-24b to attempt to maintain pressure in the body cavity at a pressure set-point. The user may set a desired pressure in the body cavity using user interface 40 (not explicitly shown). Pressure may be sensed using one or more pressure sensors in insufflator 58, in tubing 42, and/or in or on trocars 14a-14b (or other device delivering gas to a body cavity). In some embodiments, pressure may be sensed both in or on trocars 14a-14b (or some other device inserted into the body cavity) and in insufflator 58 to have redundant pressure sensing. Redundant sensors can also be within insufflator 58. Redundant sensors could be used to detect a malfunction of one of the sensors.

The gas in each input flow path is heated by at least one of the gas heaters 20a-20e and regulated by one of the pressure regulators 22a-22e. The flow from the cylinders (or house supply) may be combined to form a single flow for each output channel that runs through a conduit into flow regulation units 24a-24b for the particular output channel. Flow regulation units 24a-24b regulate the flow characteristics of the gas so as to maintain a relatively constant pressure within a body cavity into which trocars 14a-14b (or another gas delivery device such as a needle) is inserted (e.g. the peritoneal cavity). Again, all of the trocar (or other gas delivery device) options set forth in connection with FIG. 1 may be used for trocars 14a-14b and the techniques for using such trocars (or other gas delivery devices) and operating insufflator 58 discussed in connection with FIG. 1 may also be used. In addition, a needle or other device used to deliver gas to a body cavity can be used in place of trocars 14a=14b. The trocars 14a-14b (or other device) are inserted into the body cavity and deliver the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 (not explicitly shown) of insufflator 58 may control the gas from each cylinder (or house supply) to flow in a round robin fashion. Instead of combining the flow from multiple cylinders, the embodiment of FIG. 8 could instead be operated by the controller 38 to cause gas to flow from one of the cylinders (or other gas sources) 16a-16e for a first period of time (10 seconds-two minutes) and then switch the flow to a different gas cylinder 16a-16e for a second period of time (which may be the same or different than the first period of time). This can be done with how many ever cylinders (or other gas sources) are connected to the insufflator. Freeze-up may be deterred by flowing gas at a high flow rate from a single gas supply for only a short time period and then switching to a different gas supply. As discussed above, each illustrated cylinder 16 may be a plurality of cylinders joined by a splitting valve.

Another alternative would be for multichannel source switching valve 50A to alternate between cylinders 16a and 16b while multichannel source switching valve 50b alternates between cylinders 16c-16e. This would allow rotation between cylinders to deter freeze-up but would allow the flow of multiple cylinders to still be combined.

In some embodiments, rather than using a single trocar 14a-14b connected to each output channel, multiple trocars can be connected to at least one output channel of insufflator 58 using a multipath tubing set such as, for example multipath tubing set 66 described below in connection with FIG. 11. Thus either trocar 14a or trocar 14b or both could be replaced by multiple trocars and a multipath tubing set.

Controller 38 control can multichannel source switching valves 50a-50b such that input channels of valves 50a-50b are not connected to the output of the valve in question when the input is not connected to a gas source or is connected to a gas source that is malfunctioning or is almost empty. In some embodiments, multi-channel source switching valves 50a-50b may have pressure sensors on each input so that controller 38 may easily determine whether (a) a cylinder or house supply of gas or nothing is connected to each particular gas connection 36a-36e of insufflator 58, (b) a cylinder or house supply is malfunctioning as indicated by a low pressure condition, and/or (c) a cylinder is getting low on gas as indicated by declining pressure. In some embodiments, each gas input to the insufflator may pass through an inlet pressure sensor 18a-18e (not explicitly shown) to make these same determinations. Controller 38 may then control multi-channel source switching valves 50a-50b so as to not connect any particular input to the output of any of those valves where no gas source is connected to that particular input or where the gas source is malfunctioning or nearly depleted.

Insufflator 58 thus supplies insufflation gas to a body cavity and controls the flow such that insufflator 58 seeks to maintain the pressure of the body cavity at a pressure set point input by the user.

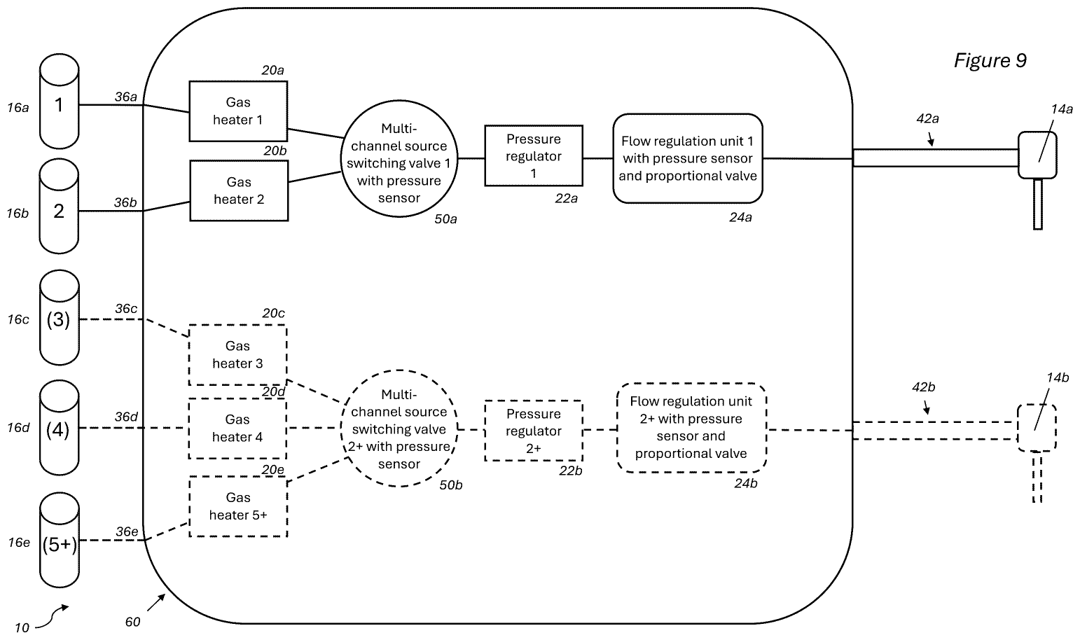

FIG. 9 illustrates another example insufflator 60 that can be used to provide high gas flows while reducing or eliminating the risk of freeze-up. FIG. 9 is similar to FIGS. 4 and 6 (as well as FIG. 3) and all of the options discussed for various components of FIGS. 4 and 6 (as well as FIG. 3) are applicable to FIG. 9 as well. Like FIG. 8, FIG. 9 illustrates a dual output port insufflator that can provide gas through separate outputs to separate trocars 14a and 14b. While this embodiment has two outputs, insufflator 60 could have any number of outputs without departing from the scope of the invention. Each output could be connected to (a) a single tank input and have the structure illustrated in FIG. 1, (b) a double tank input and have the structure illustrated at the top of FIG. 9, or (c) three or more tank inputs and have the structure illustrated at the bottom of FIG. 9. In addition, each output could have a structure like that of FIG. 1-7 or 10 feeding each output. A different structure from these figures could be chosen for each output. In addition, each output could be connected to a double tank input and have the structure illustrated at the top of FIG. 8 or three or more tank inputs and have the structure illustrated at the bottom of FIG. 8. There is no reason why one could not use a FIG. 8 channel option for one output with a FIG. 9 channel option for the other output. One could also use any of the options for FIG. 1-7 or 10 for any of the channels.

The insufflator 60 of FIG. 9, like insufflator 58 of FIG. 8, is particularly useful for use with smaller diameter trocars. Insufflator 60 has multiple outputs that provide gas flow to multiple trocars 14a-14b or other gas delivery devices such as needles, robotic hubs, gel ports, or a colonoscope. Again, while two outputs are shown, insufflator 60 could have 3, 4, 5 or more outputs. One could use structure similar to the top or bottom channels of insufflator 60 for each or some of the outputs. (Or one could use the structure similar to the top or bottom channels of insufflator 58 for each or some of the outputs or use any of the structures illustrated in FIG. 1-7 or 10).

The top output of insufflator 60 is driven by a two cylinder (or other gas source as discussed above) version of the embodiment of FIG. 4, while the bottom output of insufflator 60 is driven by a three cylinder version of the embodiment of FIG. 4. However, each output is not independent. Instead, each channel can be operated under common control of controller 38 (not explicitly shown). Insufflator 60 can advantageously control both outputs so as to maximize gas output and minimize interruption of gas flow. Ultimately, insufflator 60 controls both outputs to seek to keep the pressure inside of a body cavity as stable as possible. In most conventional insufflators, gas flow ceases temporarily so that a measurement of pressure in the abdomen can be made. The embodiment of FIG. 9 allows insufflation to be ceased by shutting off one of the on/off valves in flow regulation unit 24a or 24b. The other on/off valve in the other flow regulation unit 24a or 24b may remain open and flow can continue while the pressure is being measured, thus reducing the impact of stopping the flow to take a pressure measurement that one would normally see in a conventional insufflator. Also, because pressure measurements can be taken by both flow regulation units 24a and 24b (advantageously at different times), pressure readings can be compared to identify a bad pressure sensor and provide a degree of redundancy. Of course, either trocar 14a, trocar 14b, or both could include a pressure sensor in or on the trocar to facilitate pressure sensing at the patient so that insufflation did not have to be stopped at any time. In such an arrangement, the pressure sensors in flow regulation units 24a, 24b, could be used as backups in case of a malfunction of the pressure sensors in the trocars.

Another advantage of the embodiment illustrated in FIG. 9 is that multiple types of insufflation gas could be provided and blended together with one type of gas going to each trocar 14a and 14b. The insufflator could control the flow to each trocar so as to obtain an approximate percentage of blend of the multiple gases set by the user through the user interface.

If the embodiment of FIG. 9 uses a single gas cylinder for a channel using, for example, the structures illustrated in FIG. 1, then that channel could operate like the embodiment of FIG. 1 except that both outputs to trocars 14a and 14b would be controlled at the same time. If the embodiment of FIG. 9 uses two or more gas cylinders like the options shown at the top or bottom of FIG. 9, then each channel can be operated such that a fraction of the gas is provided by each cylinder simultaneously (e.g. all valves open for the multiple conduits of multi-channel source switching valves 50a or 50b) or each channel could be operated in a round robin fashion as described above in connection with FIGS. 4-7 such that all of the gas for a particular output to one of the trocars 14a or 14b is being provided by a single gas cylinder 16a-16e for a short period of time and then the gas would be provided by a different cylinder for the next short period of time. If a channel uses multiple cylinders, that channel could also switch to single cylinder operation where another cylinder becomes low or empty or malfunctions.

In operation, two or more gas cylinders (or a house gas supply or a mixture of cylinders and a house gas supply connections), 16a-16e are connected to one of the gas connections 36a-36e (one for each cylinder or house supply connection) for insufflator 60. When the insufflator is in operation, each cylinder 16a-16e has its own flow channel up to multichannel source switching valves 50a-50b, after which two or more of the flows may be combined to flow into one of the pressure regulators 22a-22b and flow regulation units 24a-24b, as illustrated. Insufflator 60 has two output channels but may have more output channels without departing from the scope of the invention. The flows from multiple gas sources are combined by one or more of the multichannel source switching valves 50a-50b that are capable of combining flows from multiple inputs into a single output flow. Flow regulation units 24a-24b regulate gas flow based upon sensed pressure to maintain as stable as possible a pressure in the body cavity based upon a desired pressure setting set by a user. Controller 38 (not explicitly shown) controls the flow of insufflation gas from flow regulation units 24a-24b to attempt to maintain pressure in the body cavity at a pressure set-point. The user may set a desired pressure in the body cavity using user interface 40 (not explicitly shown). Pressure may be sensed using one or more pressure sensors in insufflator 60, in tubing 42, and/or in or on trocar 14 (or other device delivering gas to a body cavity). In some embodiments, pressure may be sensed both in or on trocars 14a-14b (or some other device inserted into the body cavity) and in insufflator 60 to have redundant pressure sensing. Redundant sensors can also be within insufflator 60. Redundant sensors could be used to detect a malfunction of one of the sensors.

The gas in each input flow path is heated by at least one of the gas heaters 20a-20e. The flow from the cylinders (or house supply) may be combined to form a single flow for each output channel that runs through a conduit into flow regulation units 24a-24b for the particular output channel. Flow regulation units 24a-24b regulate the flow characteristics of the gas so as to maintain a relatively constant pressure within a body cavity into which trocars 14a-14b (or another gas delivery device such as a needle) are inserted (e.g. the peritoneal cavity). Again, all of the trocar options set forth in connection with FIG. 1 may be used for trocars 14a-14b and the techniques for using such trocars and operating insufflator 60 discussed in connection with FIG. 1 may also be used. In addition, a needle or other gas delivery device such as a robotic port, gel port, or colonoscope used to deliver gas to a body cavity can be used in place of trocars 14a-14b. The trocars 14a-14b (or other device) are inserted into the body cavity and deliver the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 (not explicitly shown) of insufflator 60 may control the gas from each cylinder (or house supply) to flow in a round robin fashion. Instead of combining the flow from multiple cylinders for a single output channel, the embodiment of FIG. 9 could instead be operated by the controller 38 to cause gas to flow from one of the cylinders (or other gas sources) 16a-16e for a first period of time (10 seconds-two minutes) and then switch the flow to a different gas cylinder 16a-16e for a second period of time (which may be the same or different than the first period of time). This can be done with all functional cylinders (or other gas sources) that are connected to the insufflator. Freeze-up may be deterred by flowing gas at a high flow rate from a single gas supply for only a short time period and then switching to a different gas supply. As discussed above, each illustrated cylinder 16 may be a plurality of cylinders joined by a splitting valve.

Another alternative would be for multichannel source switching valve 50A to alternate between cylinders 16a and 16b while multichannel source switching valve 50b alternates between cylinders 16c-16e. This would allow rotation between cylinders to deter freeze-up but would allow the flow of multiple cylinders to still be combined.

In some embodiments, rather than using a single trocar 14a-14b connected to each output channel, multiple trocars can be connected to at least one output channel of insufflator 60 using a multipath tubing set such as, for example multipath tubing set 66 described below in connection with FIG. 11. Thus either trocar 14a or trocar 14b or both could be replaced by multiple trocars and a multipath tubing set.

Controller 38 can control multichannel source switching valves 50a-50b such that input channels of valves 50a-50b are not connected to the output of the valve in question when the input is not connected to a gas source or is connected to a gas source that is malfunctioning or is almost empty. In some embodiments, multi-channel source switching valves 50a-50b may have pressure sensors on each input so that controller 38 may easily determine whether (a) a cylinder or house supply of gas or nothing is connected to each particular connection 36a-36e of insufflator 60, (b) a cylinder or house supply is malfunctioning as indicated by a low pressure condition, and/or (c) a cylinder is getting low on gas as indicated by declining pressure. In some embodiments, each gas input to the insufflator may pass through an inlet pressure sensor 18a-18e (not explicitly shown) to make these same determinations. Controller 38 may then control multi-channel source switching valves 50a-50b so as to not connect any particular input to the output of any of those valves where no gas source is connected to that particular input or where the gas source is malfunctioning or nearly depleted.

Insufflator 60 thus supplies insufflation gas to a body cavity and controls the flow such that insufflator 60 seeks to maintain the pressure of the body cavity at a pressure set point input by the user.

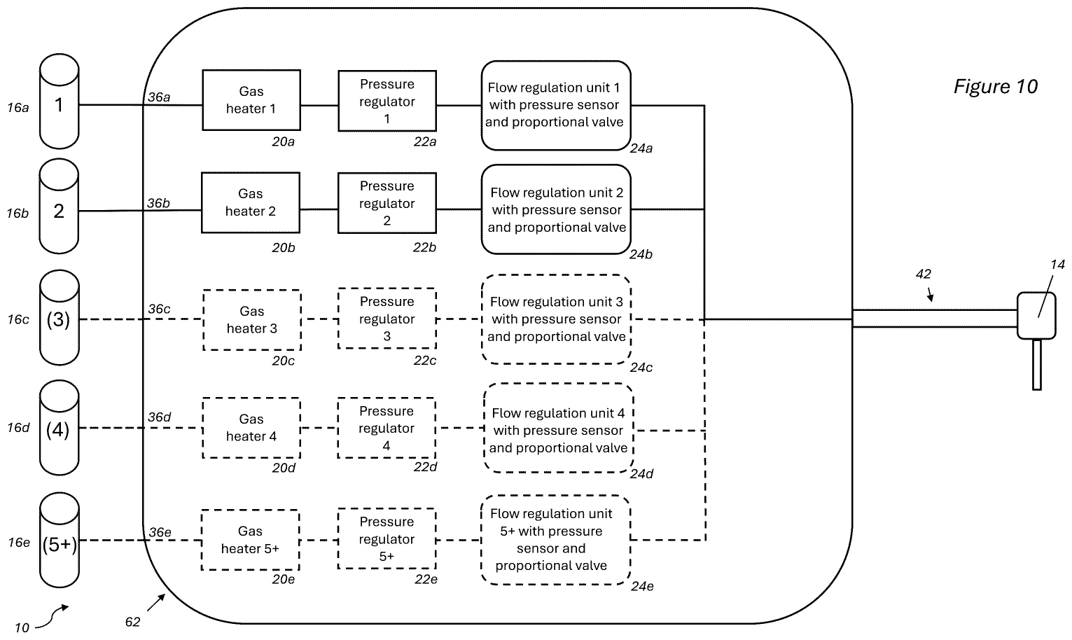

FIG. 10 illustrates another example insufflator 62 that can be used to provide high gas flows while reducing or eliminating the risk of freeze-up. In this embodiment, two or more gas cylinders (or a house supply) are used to supply gas to trocar 14. While this embodiment uses at least two gas cylinders 16a and 16b and cylinders 3-5 (16c-16e) are optional, any number of cylinders (or house supply connections) could be used without departing from the scope of the invention.

In this embodiment, each cylinder is coupled to an apparatus that is essentially the same as that illustrated in FIG. 1. A controller (not explicitly shown) controls each of the channels to achieve the desired pressure in the body cavity. The flows of each of the channels are combined using t-junctions in the conduits connected to the output of each flow regulation unit 24a-24e.

One advantage of this embodiment is that it divides the flow up among two or more cylinders, thus reducing the likelihood of freeze-up occurring. Another advantage is that if it is desired for the insufflation to be done using a mix of different insufflation gases, rather than the normal carbon dioxide, then each cylinder 16a-16e may have a different kind of insufflation gas and the controller may control the flow conditions for the gas supplied by each cylinder 16a-16e such that a mixture with approximately a desired percentage for each gas may be created. The gas mixture can be set using the user interface (not explicitly shown).

In operation, two or more gas cylinders (or a house gas supply or a mixture of cylinders and a house gas supply connections), 16a-16e are connected to one of the gas connections 36a-36e (one for each cylinder or house supply connection) for insufflator 62. When the insufflator is in operation, each cylinder (or other gas source) 16a-16e has its own flow channel up to flow regulation units 24a-24e, after which two or more of the flows may be combined in conduits with t-junctions, as illustrated. Any method of joining the conduits may be used without departing from the scope of the invention. The flows from multiple gas sources may be combined through the connection of the conduits exiting flow regulation units 24a-24e. Flow regulation units 24a-24e regulate gas flow based upon sensed pressure to maintain as stable as possible a pressure in the body cavity based upon a desired pressure setting set by a user. Controller 38 (not explicitly shown) controls the flow of insufflation gas from flow regulation units 24a-24e to attempt to maintain pressure in the body cavity at a pressure set-point. The user may set a desired pressure in the body cavity using user interface 40 (not explicitly shown). Pressure may be sensed using one or more pressure sensors in insufflator 62, in tubing 42, and/or in or on trocar 14 (or other device delivering gas to a body cavity). In some embodiments, pressure may be sensed both in or on a trocar 14 (or some other device inserted into the body cavity such as a needle, robotic hub, colonoscope, or gel port) and in insufflator 62 to have redundant pressure sensing. Redundant sensors can also be within insufflator 62. Redundant sensors could be used to detect a malfunction of one of the sensors.

The gas in each input flow path is heated by at least one of the gas heaters 20a-20e. The flow from the cylinders (or house supply) is regulated by pressure regulators 22A-22E as illustrated and the flow is regulated by flow regulation units 24a-24e. While five inputs, pressure regulators, and flow regulation units are illustrated in this embodiment, insufflator 62 could have two or more inputs whose flows are regulated individually and then combined as outputs from two or more flow regulation units 24. Flow regulation units 24a-24e regulate the collective flow characteristics of the t gas so as to maintain a relatively constant pressure within a body cavity into which trocar 14 (or another gas delivery device such as a needle) is inserted (e.g. the peritoneal cavity). Again, all of the trocar options set forth in connection with FIG. 1 may be used for trocar 14 and the techniques for using such trocars and operating insufflator 62 discussed in connection with FIG. 1 may also be used. In addition, a needle, robotic hub, colonscope, gel port, or other device used to deliver gas to a body cavity can be used in place of trocar 14. The trocar 14 (or other device) is inserted into the body cavity and delivers the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 of insufflator 62 may control the gas from each cylinder (or house supply) to flow in a round robin fashion. Instead of combining the flow from multiple cylinders for a single output channel, the embodiment of FIG. 10 could instead be operated by the controller 38 to cause gas to flow from one of the cylinders (or other gas sources) 16a-16e for a first period of time (10 seconds-two minutes) and then switch the flow to a different gas cylinder 16a-16e for a second period of time (which may be the same or different than the first period of time). This can be done with the number of cylinders (or other gas sources) that are connected to the insufflator such that flow alternates between all of them. Freeze-up may be deterred by flowing gas at a high flow rate from a single gas supply for only a short time period and then switching to a different gas supply. As discussed above, each illustrated cylinder 16 may be a plurality of cylinders joined by a splitting valve. In some embodiments, rather than using a single trocar 14 connected to the output channel, multiple trocars can be connected to the output channel of insufflator 62 using a multipath tubing set such as, for example multipath tubing set 66 described below in connection with FIG. 11.

Thus trocar 14 could be replaced by multiple trocars and a multipath tubing set.

In some embodiments, each gas input to the insufflator may pass through an inlet pressure sensor 18a-18e (not explicitly shown). Controller 38 can control valves in flow regulation units 24a-24e such that their outputs are shut off when the corresponding gas connection 36a-36e is not connected to a gas source or is connected to a gas source that is malfunctioning or is almost empty. Inlet pressure sensors 18a-18e allow controller 38 to easily determine whether (a) a cylinder or house supply of gas or nothing is connected to each particular gas connection 36a-36e of insufflator 62, (b) a cylinder or house supply is malfunctioning as indicated by a low pressure condition, and/or (c) a cylinder is getting low on gas as indicated by declining pressure. Controller 38 may then control flow regulation units 24a-24e so as to not connect any particular input to the output of any of those units where no gas source is connected to the corresponding gas connections 36a-36e or where the gas source is malfunctioning or nearly depleted.

Insufflator 62 thus supplies insufflation gas to a body cavity and controls the flow such that insufflator 62 seeks to maintain the pressure of the body cavity at a pressure set point input by the user.

FIG. 11 illustrates another example insufflator 64 that can be used to provide high gas flows while reducing or eliminating the risk of freeze-up. In this embodiment, two or more gas cylinders (or a house supply) are used to supply gas to one or more trocars 14a-14g. While this embodiment uses at least two gas cylinders 16a and 16b and cylinders 3-5 (16c-16e) are optional, any number of cylinders (or house supply connections) could be used without departing from the scope of the invention.

In this embodiment, each cylinder (or house supply connection) is coupled to an apparatus that is essentially the same as that illustrated in FIG. 1. A controller (not explicitly shown) controls each of the channels to achieve the desired pressure in the body cavity. The output of each flow regulation unit 24a-24e may feed a single trocar or multiple trocars. In this embodiment, however, the output of each flow regulation unit 24a-24e can be combined to provide a higher flow to a single trocar or group of trocars as well.

One or more valves 68a-68d may be connected to the outputs of flow regulation units 24a-24e. While four valves are shown embodiments of the invention may have zero, one, two three, or more valves. Some outputs of flow regulation units 24a-24e may be connected to valves 68a-68d while others may not. As with the embodiments discussed above, insufflator 64 may have two or more insufflation channels. The illustrated embodiment has two to five insufflation channels but could have more channels.

Valves 68a-68d are two way valves which can connect the input to one of two outputs. Output two (70A2, 70B2, 70C2, and 70D2) connects the output of a flow regulation unit 24 to a trocar 14. Controlling the valves to output through output two directs the flow of a flow regulation unit 24 to an output corresponding to that flow regulation unit. Configuration of insufflator 64 in this manner may be useful, for example, for a robotic procedure where robotic trocars have small diameters and restrict flow substantially. This insufflator configuration would allow a large gas flow to be comprised of the addition of smaller gas flows with each individual flow going to a single trocar. The controller 38 (not explicitly shown) would control the flow of each channel such that collectively the channels produced the desired pressure conditions inside of the body cavity.

Insufflator 64 is versatile, however, and can be used in procedures requiring higher gas flows through a single trocar as well. Output one (70A1, 70B1, 70C1, and 70D1) of the valves 68A-68D connect the output of a flow regulation unit through t-connections to the output of flow regulation unit 24A. Thus, the output of one or more flow regulation units 24B-24E can be combined together such that a high flow is output to one or more trocars 14a-14c connected to the insufflator's 64 first output channel. In this mode of operation, insufflator 64 would operate analogously to insufflator 62 of FIG. 10 with multiple flows combined into a single output flow. Note that insufflator 64 allows the output flows of 2, 3, 4, or all 5 (or more if so configured) channels to be combined by setting the valve 68a-68d to connect to the first output for how many ever flow regulation unit 24b-24e outputs are to be combined.

The ability to combine the flows of multiple flow regulation unit outputs or simply have each flow regulation unit 24a-24e supply gas to its own trocar (or needle or other device used to supply insufflation gas) makes insufflator 64 versatile. It can be configured to supply high flows to a single high flow trocar capable of delivering the high flows to a body cavity or it can be configured to supply lower flows to a collection of trocars capable of delivering lower flows to a body cavity in a manner that collectively provides a high flow.

FIG. 11 also shows a multipath tubing set 66 capable of delivering a high gas flow by dividing that flow among multiple trocars 14a-14c. While in this embodiment multipath tubing set 66 is fluidly coupled to three trocars, it could be coupled to one, two, or four or more trocars depending upon the flow characteristics of the trocar. Note that multipath tubing set 66 may be used with any of the embodiments illustrated in FIG. 1-10. As discussed above, some trocars are flow restrictive and may not be able to deliver gas to a body cavity at a flow rate that insufflator 64 is capable of providing. In order to take full advantage of the capabilities of insufflator 64 of providing high gas flows, it may make sense to divide the high flow that insufflator 64 can provide among multiple trocars, e.g. trocars 14a-14c. Thus, for example, if each trocar 14a-14c could only deliver 15 LPM of gas due to flow restrictions, the use of multipath tubing set 66 with three trocars 14a-14c may allow 45 LPM to be delivered as long as insufflator 64 is capable of supplying that flow and multipath tubing set 66 does not itself restrict the flow. Any of the trocar options discussed above (including in the incorporated by reference publications/applications) could be used with insufflator 64 without departing from the scope of the invention.

In some embodiments, the gas connection 36, gas heater 20, pressure regulator 22, and flow regulation unit 24a could be combined into a module. An insufflator, such as insufflator 64 could then contain one or more of the modules that are received into a chassis capable of accepting a plurality of modules. The controller 38 would have the ability to determine electronically how many modules are included in the chassis and would provide control options for insufflator 64 accordingly.

In operation, two or more gas cylinders (or a house gas supply or a mixture of cylinders and a house gas supply connections), 16a-16e are connected to one of the gas connections 36a-36e (one for each cylinder or house supply connection) for insufflator 64. When the insufflator is in operation, each cylinder 16a-16e has its own flow channel up to the output of flow regulation units 24a-24e, as illustrated. Insufflator 64 has five output channels but may have two or more output channels without departing from the scope of the invention. Insufflator 64 allows the flows from multiple gas sources to be combined by one or more of the valves 68a-68d that are capable of combining flows from multiple inputs into a single output flow. Flow regulation units 24a-24e regulate gas flow based upon sensed pressure to maintain as stable as possible a pressure in the body cavity based upon a desired pressure setting set by a user. Controller 38 (not explicitly shown) controls the collective flow of insufflation gas from flow regulation units 24a-24e to attempt to maintain pressure in the body cavity at a pressure set-point. The user may set a desired pressure in the body cavity using user interface 40 (not explicitly shown). Pressure may be sensed using one or more pressure sensors in insufflator 64, in tubing 42a-42d, 66, and/or in or on trocars 14a-14g (or other device delivering gas to a body cavity). In some embodiments, pressure may be sensed both in or on trocars 14a-14g (or some other device inserted into the body cavity) and in insufflator 64 to have redundant pressure sensing. Redundant sensors can also be within insufflator 64. Redundant sensors could be used to detect a malfunction of one of the sensors.

The gas in each input flow path is heated by at least one of the gas heaters 20a-20e. The flow from the cylinders (or house supply) may be combined to form a single flow for the first output channel or each output channel can have its own individual flow. Using insufflator 64 to have each of the five output channels (or 2 or 3 or 4 or more output channels) have its own flow may be particularly useful in a robotic system with flow restricted s where multiple flows in parallel are needed to provide a cumulative flow that is adequate to handle large leaks during a surgery. Flow regulation units 24a-24e regulate the flow characteristics of the gas so as to maintain a relatively constant pressure within a body cavity into which trocars 14a-14g (or another gas delivery device such as a needle, robotic hub, colonscope, or gel port) are inserted (e.g. the peritoneal cavity). Again, all of the trocar options set forth in connection with FIG. 1 may be used for trocars 14a-14g and the techniques for using such trocars and operating insufflator 64 discussed in connection with FIG. 1 may also be used. In addition, a needle, robotic hub, colonscope, gel port, or other device used to deliver gas to a body cavity can be used in place of one or more of trocars 14a-14g. The trocars 14a-14g (or other device) are inserted into the body cavity and deliver the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 (not explicitly shown) of insufflator 64 may control the gas from each cylinder (or house supply) to flow in a round robin fashion. Instead of flowing gas through multiple outputs at the same time, the embodiment of FIG. 11 could instead be operated by the controller 38 to cause gas to flow from one of the cylinders (or other gas sources) 16a-16e for a first period of time (10 seconds-two minutes) and then switch the flow to a different gas cylinder 16a-16e for a second period of time (which may be the same or different than the first period of time). This can be done with the number of cylinders (or other gas sources) that are connected to the insufflator such that all cylinders or other gas sources supply gas in a round robin fashion. Freeze-up may be deterred by flowing gas at a high flow rate from a single gas supply for only a short time period and then switching to a different gas supply. As discussed above, each illustrated cylinder 16 may be a plurality of cylinders joined by a splitting valve.

In some embodiments, rather than using a single trocar 14 connected to each output channel, multiple trocars can be connected to at least one output channel of insufflator 64 using a multipath tubing set such as, for example multipath tubing set 66 described above. Thus any of the trocars 14d-14g could be replaced by multiple trocars and a multipath tubing set.

In some embodiments, each gas input to the insufflator may pass through an inlet pressure sensor 18a-18e (not explicitly shown). Controller 38 can control valves in flow regulation units 24a-24e such that their outputs are shut off when the corresponding gas connection 36a-36e is not connected to a gas source or is connected to a gas source that is malfunctioning or is almost empty. Inlet pressure sensors 18a-18e allow controller 38 to easily determine whether (a) a cylinder or house supply of gas or nothing is connected to each particular gas connection 36a-36e of insufflator 62, (b) a cylinder or house supply is malfunctioning as indicated by a low pressure condition, and/or (c) a cylinder is getting low on gas as indicated by declining pressure. Controller 38 may then control flow regulation units 24a-24e so as to not connect any particular input to the output of any of those units where no gas source is connected to the corresponding gas connections 36a-36e or where the gas source is malfunctioning or nearly depleted.

Insufflator 64 thus supplies insufflation gas to a body cavity and controls the flow such that insufflator 64 seeks to maintain the pressure of the body cavity at a pressure set point input by the user.

Valves 68a-68d could be omitted and insufflator 64 could simply be a multiple output channel insufflator where controller 38 controls the flow in each of the channels simultaneously to seek to achieve stable pressure inside of a body cavity at a pressure point set by a user of insufflator 64. As discussed above, such an insufflator may have 2, 3, 4, 5, or more output channels. Such an insufflator reduces the possibility of freeze-up by dividing the supply of insufflation gas among a plurality of gas sources.

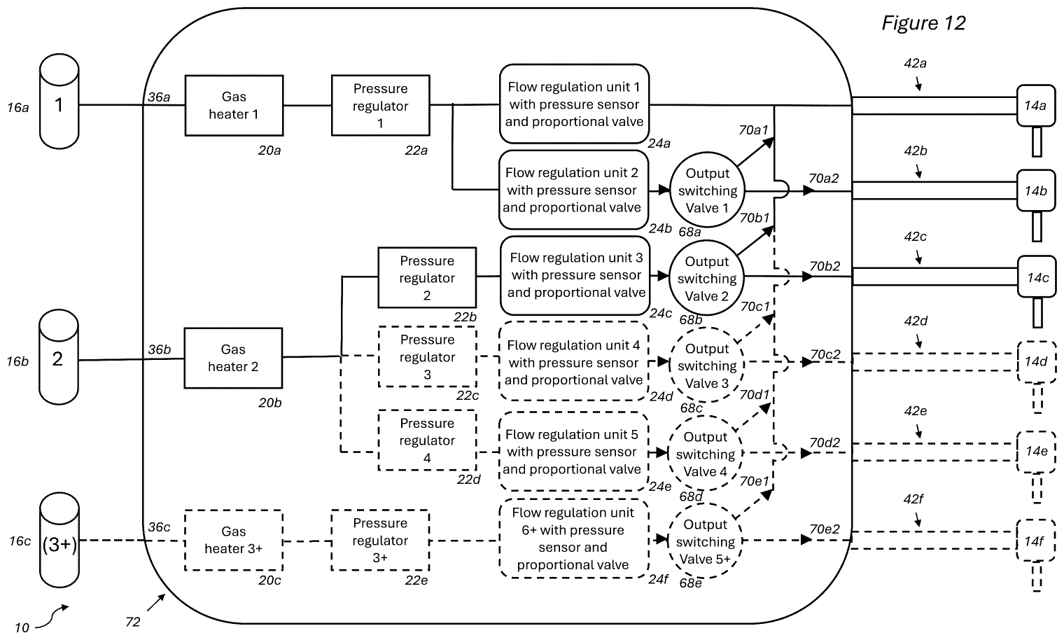

FIG. 12 illustrates another example insufflator 72 that can be used to provide high gas flows while reducing or eliminating the risk of freeze-up. In this embodiment, two or more gas cylinders (or a house supply) are used to supply gas to one or more trocars 14a-14f. While this embodiment uses at least two gas cylinders 16a and 16b and cylinder 3 (16c) is optional, any number of cylinders (or house supply connections) could be used without departing from the scope of the invention.

In this embodiment, each cylinder (or house supply connection) is coupled to an apparatus that feeds one or more output channels. A controller (not explicitly shown) controls each of the output channels to achieve the desired pressure in the body cavity. The output of each flow regulation unit 24a-24f may feed a single trocar or multiple trocars. In this embodiment, however, the output of each flow regulation unit 24a-24f can be combined to provide a higher flow to a single trocar or group of trocars as well.

One or more valves 68a-68e may be connected to the outputs of flow regulation units 24a-24f. While five valves are shown, embodiments of the invention may have zero, one, two three, or more valves. Some outputs of flow regulation units 24a-24f may be connected to valves 68a-68e while others may not. As with the embodiments discussed above, insufflator 72 may have two or more output insufflation channels. The illustrated embodiment has three to six insufflation channels but could have more or less channels.

Valves 68a-68e are two way valves which can connect the input to one of two outputs. Output two (70a2, 70b2, 70c2, 70d2 and 70e2) connects the output of a flow regulation unit 24 to a trocar 14. Controlling the valves to output through output two directs the flow of a flow regulation unit 24 to an output corresponding to that flow regulation unit. Configuration of insufflator 72 in this manner may be useful, for example, for a robotic procedure where robotic trocars have small diameters and restrict flow substantially. This insufflator configuration would allow a large gas flow to be comprised of the addition of smaller gas flows with each individual flow going to a single trocar. The controller 38 (not explicitly shown) would control the flow of each output channel such that collectively the channels produced the desired pressure conditions inside of the body cavity.

Insufflator 72 is versatile, however, and can be used in procedures requiring higher gas flows through a single trocar as well. Output one (70A1, 70B1, 70C1, 70D1 and 70E1) of the valves 68A-68E connect the output of a flow regulation unit through t-connections to the output of flow regulation unit 24a. Thus, the output of one or more flow regulation units 24b-24f can be combined together such that a high flow is output to one or more trocars 14a connected to the insufflator's 72 first output channel. In this mode of operation, insufflator 72 would operate analogously to insufflator 62 of FIG. 10 with multiple flows combined into a single output flow. Note that insufflator 72 allows the output flows of 2, 3, 4, 5, or all 6 (or more if so configured) output channels to be combined by setting the valve 68a-68e to connect to the first output for whichever flow regulation unit 24b-24f outputs are to be combined.

The ability to combine the flows of multiple flow regulation unit outputs or simply have each flow regulation unit 24a-24f supply gas to its own trocar (or needle or other device used to supply insufflation gas) makes insufflator 72 versatile. It can be configured to supply high flows to a single high flow trocar capable of delivering the high flows to a body cavity or it can be configured to supply multiple flows to a collection of trocars capable of delivering flows to a body cavity in a manner that collectively provides a high flow.

Another option is to use a multipath tubing set, such as multipath tubing set 66 of FIG. 11 (or another tubing set having more or less branches), to deliver a high gas flow. Where insufflator 72 directs a high gas flow to one of the output channels, the multi-path tubing set may be capable of delivering a high gas flow by dividing that flow among multiple trocars 14a-14c. While in the FIG. 11 embodiment multipath tubing set 66 is fluidly coupled to three trocars, it could be coupled to one, two, or four or more trocars depending upon the flow characteristics of the trocar. As discussed above, some trocars flow restrictive and may not be able to deliver gas to a body cavity at a flow rate that insufflator 72 is capable of providing. In order to take full advantage of the capabilities of insufflator 72 of providing high gas flows, it may make sense to divide the high flow that insufflator 72 can provide among multiple trocars, e.g. trocars 14a-14c of FIG. 11. Thus, for example, if each trocar 14a-14c could only deliver 15 LPM of gas due to flow restrictions, the use of multipath tubing set 66 with three trocars 14a-14c as illustrated in FIG. 11 may allow 45 LPM to be delivered as long as insufflator 72 is capable of supplying that flow and multipath tubing set 66 does not itself restrict the flow. Any of the trocar options discussed above (including in the incorporated by reference publications/applications) could be used with insufflator 72 without departing from the scope of the invention.

In some embodiments, the gas connection 36, gas heater 20, pressure regulator 22, and flow regulation unit 24a could be combined into a module. An insufflator, such as insufflator 72 could then contain one or more of the modules that are received into a chassis capable of accepting a plurality of modules. The controller 38 would have the ability to determine electronically how many modules are included in the chassis and would provide control options for insufflator 72 accordingly.

Three different options are illustrated in FIG. 12 for the flow path between a gas connection 36a-36c and one or more output channels. Insufflator 72 could have identical structure between each gas connection 36a-36c and the one or more outputs associated with that gas connection or could have different structure for each channel. While three options for the structure are illustrated in FIG. 12, other options could be used without departing from the scope of the invention. Any option could be used that connects a gas connection 36 to one or more outputs. FIG. 12 illustrates an architecture for an insufflator 72 that may use two or more gas connections 36 each of which connect to one or more insufflator output channels. Any structure capable of handling both cylinders and other gas supplies (such as a house gas supply) and regulating the pressure inside of a body cavity using feedback control can be used.

In FIG. 12, for example, the bottom output channel is connected to a structure that is essentially the same as the structure of FIG. 1. However, all of the flow regulation units 24a-24g may be controlled simultaneously by controller 38 so as to provide a relatively stable pressure in a body cavity based upon a pressure set point. Feedback control using pressure sensor as described above may be used by controller 38 to maintain the relatively stable pressure. The top two output channels in FIG. 12 are connected to a single gas input 36a which has a conduit that splits into two conduits after pressure regulator 22a to feed multiple flow regulation units 24a-24b. Of course, the flow could split before heater 20a and an additional heater and pressure regulator could be provided. The third-fifth output channels illustrated in FIG. 12 are fed by a single gas cylinder 16b (or other gas supply such as a house supply). In this example, the flow from cylinder 16b splits after the heater 20b but could split before the heater or after the pressure regulator.

In operation, two or more gas cylinders (or a house gas supply or a mixture of cylinders and a house gas supply connections), 16a-16c are connected to one of the gas connections 36a-36c (one for each cylinder or house supply connection) for insufflator 72. When the insufflator is in operation, each cylinder 16a-16c may be connected to one or more output channels and be fluidly connected to one or more trocars 14a-14f or other gas delivery devices. Insufflator 72 has six output channels but may have two or more output channels without departing from the scope of the invention. Insufflator 72 allows the flows from multiple gas sources to be combined by one or more of the valves 68a-68e that are capable of combining flows from multiple inputs into a single output flow. Flow regulation units 24a-24f regulate gas flow based upon sensed pressure to maintain as stable as possible a pressure in the body cavity based upon a desired pressure setting set by a user. Controller 38 (not explicitly shown) controls the collective flow of insufflation gas from flow regulation units 24a-24f to attempt to maintain pressure in the body cavity at a pressure set-point. The user may set a desired pressure in the body cavity using user interface 40 (not explicitly shown). Pressure may be sensed using one or more pressure sensors in insufflator 72, in tubing 42a-42f, multipath tubing set 66 (not explicitly shown), and/or in or on trocars 14a-14f (or other device delivering gas to a body cavity). In some embodiments, pressure may be sensed both in or on trocars 14a-14f (or some other device inserted into the body cavity) and in insufflator 72 to have redundant pressure sensing. Redundant sensors can also be within insufflator 72. Redundant sensors could be used to detect a malfunction of one of the sensors.

The gas in each input flow path is heated by at least one of the gas heaters 20a-20c. The flow from the cylinders (or house supply) may be combined to form a single flow for the first output channel or each output channel can have its own individual flow. Using insufflator 72 to have each of the six output channels (or 2, 3, 4 or 5 or more output channels) have its own flow may be particularly useful in a robotic system with flow restricted trocars where multiple flows in parallel are needed to provide a cumulative flow that is adequate to handle large leaks during a surgery. Flow regulation units 24a-24f regulate the flow characteristics of the gas so as to maintain a relatively constant pressure within a body cavity into which trocars 14a-14f (or another gas delivery device such as a needle, robotic hub, colonscope, or gel port) are inserted (e.g. the peritoneal cavity). Again, all of the trocar options set forth in connection with FIG. 1 may be used for trocars 14a-14f and the techniques for using such trocars and operating insufflator 72 discussed in connection with FIG. 1 may also be used. In addition, a needle, robotic hub, colonscope, gel port, or other device used to deliver gas to a body cavity can be used in place of one or more of trocars 14a-14f. The trocars 14a-14f (or other device) are inserted into the body cavity and deliver the insufflation gas to the body cavity. In this embodiment, carbon dioxide is delivered to the body cavity but other insufflation gasses (e.g helium) or a mix of insufflation gasses could be used without departing from the scope of the invention.

In an alternative embodiment, controller 38 (not explicitly shown) of insufflator 72 may control the gas from each cylinder (or house supply) to flow in a round robin fashion. Instead of flowing gas through multiple outputs at the same time, the embodiment of FIG. 12 could instead be operated by the controller 38 to cause gas to flow from one of the cylinders (or other gas sources) 16a-16c for a first period of time (10 seconds-two minutes) and then switch the flow to a different gas cylinder 16a-16c for a second period of time (which may be the same or different than the first period of time). This can be done with the number of cylinders (or other gas sources) that are connected to the insufflator such that all cylinders or other gas sources supply gas in a round robin fashion. Freeze-up may be deterred by flowing gas at a high flow rate from a single gas supply for only a short time period and then switching to a different gas supply. As discussed above, each illustrated cylinder 16 may be a plurality of cylinders joined by a splitting valve.

In some embodiments, rather than using a single trocar 14 connected to each output channel, multiple trocars can be connected to at least one output channel of insufflator 72 using a multipath tubing set such as, for example multipath tubing set 66 described above. Thus any of the trocars 14a-14f could be replaced by two or more trocars (or other gas delivery devices) and a multipath tubing set have 2 or more branches.

In some embodiments, each gas input to the insufflator may pass through an inlet pressure sensor 18a-18c (not explicitly shown). Controller 38 can control valves in flow regulation units 24a-24f such that their outputs are shut off when the corresponding gas connection 36a-36c is not connected to a gas source or is connected to a gas source that is malfunctioning or is almost empty. Inlet pressure sensors 18a-18c allow controller 38 to easily determine whether (a) a cylinder or house supply of gas or nothing is connected to each particular gas connection 36a-36e of insufflator 72, (b) a cylinder or house supply is malfunctioning as indicated by a low pressure condition, and/or (c) a cylinder is getting low on gas as indicated by declining pressure. Controller 38 may then control flow regulation units 24a-24f so as to not connect any particular input to the output of any of those units where no gas source is connected to the corresponding gas connections 36a-36c or where the gas source is malfunctioning or nearly depleted.

Insufflator 72 thus supplies insufflation gas to a body cavity and controls the flow such that insufflator 72 seeks to maintain the pressure of the body cavity at a pressure set point input by the user.

Valves 68a-68e could be omitted and insufflator 72 could simply be a multiple output channel insufflator where controller 38 controls the flow in each of the channels simultaneously to seek to achieve stable pressure inside of a body cavity at a pressure point set by a user of insufflator 72. As discussed above, such an insufflator may have 2, 3, 4, 5, or more output channels. Such an insufflator reduces the possibility of freeze-up by dividing the supply of insufflation gas among a plurality of gas sources.

As discussed herein, embodiments 2-12 could be used with a house gas supply. One option for using these embodiments with a house gas supply is to use a conduit between the port on the wall for the house gas supply and the insufflator that splits into two or more conduits to connect to the two or more gas inputs for the insufflator. However, gas supplied by a house supply tends to be at a lower pressure and freeze up issues are greatly reduced or eliminated by using a house supply. Thus, the embodiments of FIGS. 2-12 may operate with the house supply connected to a single gas connection 36 of the insufflator. In such an example, the insufflator would be configured to obtain all the gas from the house connection. For example, in the FIG. 2 embodiment, the house gas could be connected to any one of the gas inputs 36a-36e and make use of a single gas heater 20a-20e and single pressure regulator 22a-22e. Valves may be closed as discussed in connection with FIG. 2 to prevent gas from flowing out of gas connections 36a-36e that are not connected to any gas source. In FIG. 3, the house connection could be connected to one or more of the gas connections 36a-36e, again making use of valves to prevent gas from flowing out of a gas connection 36a-36e that remains unconnected.

In FIGS. 4-7, the house gas connection could be connected to one or more of the gas connections 36a-36e. Controller 38 would simply configure the multichannel source switching valve or valves 50 to channel gas from the house connection to the remainder of the insufflator. The multichannel source switching valves would be controlled by controller 38 so as to prevent gas from flowing back out of a gas connection 36a-36f that is not connected to a gas source. In the FIG. 8-9, it may be desirable to connect the house connection to at least one connection of each of the multichannel source switching valves 50a-50b in FIGS. 8-9 using some type of hose that connects to the house supply and then splits into two flow paths. This would allow use of both output channels for insufflator 58. Again, the multichannel source switching valves 50 may be configured by the controller 38 to close off inputs where there is no gas connection and allow the gas to flow from the house gas supply to provide flow to each of the trocars 14a-14b.

In the FIG. 10 embodiment, the house supply could be connected to one or more of the gas connections 36a-36e. If trocar 14 is a high flow trocar then it may be able to supply a high flow by itself.

In the FIG. 11-12 embodiments, one might only connect a house supply to one or more gas connections 36a-36e, such as gas connection 36a, if a high flow trocar 14 (or other gas delivery device) is being used or if a multipath tubing set 66 is being used. If, on the other hand, multiple trocars 14 (or other gas delivery devices) are flow restrictive and are being used to deliver a piece of a high flow so that collectively a high flow is delivered, then it may be desirable to connect the house source to multiple gas connections 36a-36e.

Any of the illustrated embodiments may include a smoke evacuation capability. In some embodiments, the insufflator may include a dedicate smoke evacuation channel that comprises a tubing set connected to the body cavity and is also connected to a source of suction within the insufflator. In such an embodiment, a filter may be provided to filter the smoke or a connection may be made to a house suction port to remove the smoke from the operating room. In other embodiments, the insufflator may use one of the insufflation channels sometimes for insufflation and sometimes for smoke evacuation. Any of the insufflation channels in any of the embodiments could be configured to include the smoke evacuation apparatus and valving of U.S. patent application Ser. No. 17/058,752, which was published as Pub. No. US 2021/0213214, which is hereby incorporated by reference as if fully set forth herein. The flue gas outlet of an insufflator so configured could be (a) connected to a filter that removes toxic chemicals from surgical gas/smoke and vents the gas to the operating room, (b) connected to a house suction line that removes the gas/smoke to a location where it can be safely disposed of, and/or (c) connected to a cannister that collects the gas/smoke for later disposal.

Any of the illustrated embodiments may include the ability to heat and humidify the insufflation gas. Heating and humidification may occur within the insufflator, in the tubing 42 or multipath tubing set 66, or at the trocar 14. In some embodiments, heating and humidification may be provided by a separate device such as the Insuflow device sold by Lexion Medical.

The embodiments of FIGS. 8-12 used a method of insufflating a body cavity that differs from that of FIGS. 1-7 in that FIGS. 8-12 employ two or more flow regulation units working together. Each of the insufflators of FIGS. 8-12 receive a first stream of gas from a first gas source coupled to a first gas input connection of the insufflator. Each insufflator also receives a second stream of gas from a second gas source coupled to a second gas input connection of the insufflator. Of course only a single gas stream could be used if other gas streams are unavailable, such as, for example, if a gas source malfunctions or is being changed out because it is near empty or empty.

Continuing with the method, each of the insuffulators may be fluidly coupled to a body cavity. That coupling may include a conduit joined to a gas delivery device, such as any of the gas delivery device options discussed above in connection with FIG. 1 (e.g. a trocar, needle, colonoscope, gel port, robotic hub). The insufflator may regulate the flow of gas to the body cavity with at least a first flow regulation unit coupled to a first gas input connection and a second flow regulation unit coupled to a second gas input connection. The first and second flow regulation units may be further coupled to the body cavity to supply gas thereto, wherein the first and second flow regulation units regulate the flow of gas to the body cavity in response to pressure measurements equal or proportional to the pressure in the body cavity to which the insufflator is supplying gas.

In some embodiments, the first and second flow regulation units may be connected to the body cavity through separate conduits leading to at least one gas delivery device (e.g. FIGS. 8, 9, 11 and 12). In other embodiments, the output of the first and second flow regulation units may be joined internally within the insufflator and delivered through a conduit connected to a single output of the insufflator (e.g. FIGS. 10, 11, and 12).

In a method using the FIG. 8 or FIG. 9 embodiments, the insufflator has multiple outputs, each connected to a flow regulation unit. The output of each flow regulation unit may be connected via an output of the insufflator through at least one conduit (42a/42b) to one or more gas delivery devices. All of the options discussed in connection with FIG. 1 for gas delivery devices may be used. Also, one or more of the outputs of the insufflator may be connected to a multipath tubing set including all the options discussed above in connection with FIG. 11.

In a method using the FIG. 10 embodiment, the output of the first and second flow regulation units may be joined together inside of the insufflator, e.g. using flow channels joined by t-junctions, and output through a single output channel. All of the options discussed in connection with FIG. 1 for gas delivery devices may be used. Also, one or more of the outputs of the insufflator may be connected to a multipath tubing set including all the options discussed above in connection with FIG. 11.

In a method using the FIG. 11 or FIG. 12 embodiments, the outputs of the first and second flow regulation units may either be joined together inside of the insufflator by making use of valves 68a-68d or the insufflator may have multiple outputs, each connected to a flow regulation unit. All of the options discussed in connection with FIG. 1 for gas delivery devices may be used to deliver gas to a body cavity when coupled to at least one output of the insufflator. Also, one or more of the outputs of the insufflator may be connected to a multipath tubing set including all the options discussed above in connection with FIG. 11.

The method may include, such as in FIG. 8, connecting more gas cylinders (or other gas supplies) to the insufflator, with each gas source feeding a pressure regulator with the output of multiple pressure regulators joined to feed the input of one of the flow regulation units. The method may also include, such as in FIG. 9, joining the flow from two or more gas cylinders (or other gas supplies) to the insufflator prior to a pressure regulator which is coupled to one of the flow regulation units. The method may also include, such as in FIG. 10 and FIG. 11, connecting two or more gas cylinders (or other gas suppliers) to the insufflator, with each gas source feeding a pressure regulator and the output of each pressure regulator feeding a dedicated flow regulation unit. The method may also include, such as in the top gas input in FIG. 12, connecting a gas cylinder to a pressure regulator whose output feeds two or more flow regulation units. A mixture of any of the above methods of flow regulation may be used without departing from the scope of the invention.

In some embodiments, particularly those where a multi-channel source switching valve is used, the method may include outputting a stream of gas that includes gas from multiple cylinders (or other gas supplies) connected to the insufflator. In other embodiments, the method may include outputting a stream of gas the alternates between gas from one or more cylinders (or other gas supplies) connected to the insufflator and a different one or more cylinders (or other gas supplies) as described above. In other words, the method of operation may involve the insufflator combining the flow from multiple cylinders or using the flow from fewer than all connected cylinders at any one time and then changing the cylinder from which flow is being used to supply the body cavity.

Although the embodiments in the disclosure have been described in detail, numerous changes, substitutions, variations, alterations, and modifications may be ascertained by those skilled in the art. It is intended that the present disclosure encompass all such changes, substitutions, variations, alterations, and modifications. Additionally, one or more features described herein may be combined with one or more embodiments also described herein.

To aid the Patent Office, and any readers of any patent issued on this application in interpreting the claims appended hereto, it is noted that applicant does not intend any of the appended claims to invoke 35 U.S.C. § 112 (f) as it exists on the date of filing unless the words “means for” or “step for” are used in the particular claim. Furthermore, none of the description in the present application should be read as implying that any particular element, step, or function is an essential element which must be included in the claim scope. The scope of the patented subject matter is defined only by the allowed claims. Thus, the extent of legal protection will be determined by the limitations recited in the allowed claims and their equivalents. Unless explicitly recited, other aspects of the present disclosure as described in this specification do not limit the scope of the claims.

What is claimed is:

1. An insufflator comprising:
at least a first and second gas input connection, the first and second gas input connections operable to be connected to a first gas source and a second gas source;
a first gas output connection;
at least a first flow regulation unit coupled to the first and second gas input connection and to the first gas output, the first flow regulation unit configured to regulate the flow of gas through the first gas output connection in response to pressure measurements equal or proportional to the pressure in a body cavity to which the insufflator is supplying gas.

2. The insufflator of claim 1, further comprising:
a first pressure regulator having an input coupled to the first gas input connection and an output coupled to the flow regulation unit; and
a second pressure regulator having an input coupled to the second gas input connection and an output coupled to the flow regulation unit.

3. The insufflator of claim 2,
wherein the output of the first pressure regulator and the output of the second pressure regulator are fluidly coupled to a first conduit coupled to the first flow regulation unit.

4. The insufflator of claim 2, further comprising:
a multi-channel source switching valve having at least a first input, a second input, and first output; and
wherein the output of the first pressure regulator is coupled to the first input of the multi-channel source switching valve;
wherein the output of the second pressure regulator is coupled to the second input of the multi-channel source switching valve; and
wherein and the output of the multi-channel source switching valve is fluidly coupled to a first conduit coupled to the first flow regulation unit.

5. The insufflator of claim 1, further comprising:
a heater;
wherein the heater is configured to heat gas entering the insufflator through the first gas input connection prior to the gas reaching the first flow regulation unit.

6. A method of insufflation of a body cavity,
comprising:
receiving a first stream of gas from a first gas source coupled to a first gas input connection of an insufflator;
receiving a second stream of gas from a second gas source coupled to a second gas input connection of the insufflator;
fluidly coupling the insufflator to a body cavity;
regulating the flow of gas to the body cavity with a first flow regulation unit coupled to the first and second gas input connections and further coupled to the body cavity to supply gas thereto, wherein the first flow regulation unit regulates the flow of gas to the body cavity in response to pressure measurements equal or proportional to the pressure in the body cavity to which the insufflator is supplying gas.

7. The method of claim 6,
wherein the insufflator further comprises a first pressure regulator having an input coupled to the first gas input connection and an output coupled to the flow regulation unit; and
wherein the insufflator further comprises a second pressure regulator having an input coupled to the second gas input connection and an output coupled to the flow regulation unit.

8. The method of claim 7,
wherein the output of the first pressure regulator and the output of f the second pressure regulator are fluidly coupled to a first conduit coupled to the first flow regulation unit.

9. The method of claim 7,
wherein the insufflator further comprises a multi-channel source switching valve having at least a first input, a second input, and first output;
wherein the output of the first pressure regulator is coupled to the first input of the multi-channel source switching valve;
wherein the output of the pressure regulator is coupled to the second input of the multi-channel source switching valve; and
wherein and the output of the multi-channel source switching valve is fluidly coupled to a first conduit coupled to the first flow regulation unit.

10. The method of claim 6,
wherein the first gas source comprises a gas cylinder and wherein the second gas source comprises a gas cylinder.

11. The method of claim 6,
wherein the flow regulation unit receives at least some of the gas in the first stream of gas and at least some of the gas in the second stream of gas in a combined flow.

12. The method of claim 6,
wherein the flow regulation unit receives an alternating flow of at least some of the gas in the first stream of gas, then at least some of the gas in the second stream of gas and then back to at least some of the gas in the first stream of gas.

13. The method of claim 9, further comprising
operating the multi-channel source switching valve such that its first input and its second input are coupled to its output simultaneously such that at least some of the first stream of gas and at least some of the second stream of gas are combined at the output of the multi-channel source switching valve.

14. The method of claim 9, further comprising:
operating the multi-channel source switching valve such that its first input and its second input are coupled to its output in an alternating fashion such that the second input of the multi-channel source switching valve is closed when the first input of the multi-channel source switching valve is open and vice versa.

15. The method of claim 6,
wherein at least some of the pressure measurements are made using a pressure sensor within the insufflator.

16. The method of claim 6,
wherein at least some of the pressure measurements are made using a pressure sensor within or on a gas delivery device inserted into the body cavity.

17. The method of claim 16,
wherein the gas delivery device comprises a trocar.

18. The method of claim 6, further comprising:
fluidly connecting the insufflator to a body cavity using a tubing set with a plurality of branches, where at least two of the plurality of branches are coupled, respectively, to two different gas delivery devices inserted into the body cavity.

19. The method of claim 13,
wherein the first gas source comprises a gas cylinder and wherein the second gas source comprises a gas cylinder.

20. The method of claim 14,
wherein the first gas source comprises a gas cylinder and wherein the second gas source comprises a gas cylinder.
